(19) United States
(12) Patent Application Publication
(10) Pub. No.: US 2025/0104299 A1
(43) Pub. Date: Mar. 27, 2025

(54) NEURAL COMPUTED TOMOGRAPHY RECONSTRUCTION

(71) Applicant: THE REGENTS OF THE UNIVERSITY OF CALIFORNIA, Oakland, CA (US)

(72) Inventors: Francisco Contijoch, San Diego, CA (US); Kunal Gupta, Pittsburgh, PA (US); Brendan Colvert, Yorba Linda, CA (US)

(21) Appl. No.: 18/728,259

(22) PCT Filed: Jan. 13, 2023
(86) PCT No.: PCT/US2023/060680

§ 371 Date: Jul. 11, 2024

Related U.S. Application Data
(60) Provisional application No. 63/299,344, filed on Jan. 13, 2022.

(51) Int. Cl. G06T 11/00; G06V 10/26; G06V 10/764; G06V 10/774; G06V 10/82

(52) U.S. Cl. CPC G06T 11/006; G06T 11/008; G06V 10/26; G06V 10/764; G06V 10/774; G06V 10/82; G06T 2210/41; G06T 2211/441; G06V 2201/03

(57) ABSTRACT

Methods and systems that pertain to an image reconstruction of motion-corrupted images are disclosed. In some embodiments of the disclosed technology, an image reconstruction method includes obtaining an initial estimate of object boundaries from motion-corrupted images, creating an implicit representation of the motion-corrupted images, updating the implicit representation of the motion-corrupted images using acquired imaging data to generate an updated implicit representation of the motion-corrupted images, and converting the updated implicit representation of the motion corrupted images to an explicit set of motion-corrected images.

### CROSS-REFERENCE TO RELATED APPLICATION

This patent document claims priority to and benefits of U.S. Provisional Appl. No. 63/299,344, entitled “NEURAL COMPUTED TOMOGRAPHY RECONSTRUCTION” and filed on Jan. 13, 2022. The entire contents of the before-mentioned patent application are incorporated by reference as part of the disclosure of this document.

### STATEMENT REGARDING FEDERALLY SPONSORED RESEARCH OR DEVELOPMENT

This invention was made with government support under HL143113 awarded by the National Institutes of Health (NIH). The government has certain rights in the invention.

### TECHNICAL FIELD

The invention relates to methods and devices that pertain to image reconstruction technology.

### BACKGROUND

X-ray computed tomography (CT)'s ability to noninvasively and volumetrically evaluate patient anatomy with high spatial resolution has led to a widespread clinical use. Unfortunately, image quality can be reduced if object motion occurs during the acquisition. This is particularly concerning when imaging fast moving structures (e.g., the heart). As a result, while CT is used for the evaluation of suspected coronary artery disease and acute chest pain, even small motions lead to significant blurring of key structures.

### SUMMARY

The disclosed technology can be implemented in some embodiments to provide methods, materials and devices that pertain to an image reconstruction such as a neural computed tomography (CT) reconstruction.

In some implementations of the disclosed technology, an image reconstruction method includes obtaining an initial estimate of object boundaries from motion-corrupted images; creating an implicit representation of the motion-corrupted images; updating the implicit representation of the motion-corrupted images using acquired imaging data to generate an updated implicit representation of the motion-corrupted images; and converting the updated implicit representation of the motion corrupted images to an explicit set of motion-corrected images.

In some implementations of the disclosed technology, an image reconstruction method includes obtaining an implicit signed distance function image of an object; updating the implicit signed distance function image to produce an updated implicit signed distance function image that matches acquired imaging data; and converting the updated implicit signed distance function image into an explicit signed distance function image of the object.

In some implementations of the disclosed technology, an image reconstruction method includes performing an image segmentation on an initial reconstructed image to obtain binary classification images for identifying an object of interest in the initial reconstructed image; converting the binary classification images into a first set of signed distance function (SDF) images configured to explicitly represent the object of interest; converting the first set of signed distance function into a second set of SDF images configured to implicitly represent the object of interest; training the second set of SDF images to match acquired sinogram data; and converting the trained second set of SDF images into a third set of SDF images configured to explicitly represent the object of interest.

In some implementations of the disclosed technology, an image reconstruction method includes performing a first explicit-to-implicit representation conversion to obtain an implicit signed distance function image of an object by: obtaining an initial binary classification image of the object; and converting the binary classification image into the implicit distance function image; performing a first training operation on the implicit signed distance function image by updating the implicit signed distance function image to produce a first updated implicit signed distance function image that matches an acquired sinogram; performing a first implicit-to-explicit representation conversion from the updated implicit signed distance function image to generate a first binary image; performing a second explicit-to-implicit representation conversion by feeding the first binary image as the binary classification image of the object; and performing a second training operation and a second implicit-to-explicit representation conversion to generate a second binary image.

The above and other aspects and implementations of the disclosed technology are described in more detail in the drawings, the description and the claims.

### BRIEF DESCRIPTION OF THE DRAWINGS

FIG. 1 shows an example of a neural computed tomography algorithm implemented based on some embodiments of the disclosed technology.

FIG. 2 shows filtered back projection (FBP) reconstructed images and images generated by a neural computed tomography algorithm implemented based on some embodiments of the disclosed technology.

FIGS. 3A and 3B show an example of a neural computed tomography algorithm reconstruction of an object based on some embodiments of the disclosed technology.

FIG. 4 shows an example of a neural computed tomography algorithm implemented based on some embodiments of the disclosed technology that improves single and multiple gantry rotation imaging.

FIG. 5 shows evaluation of the neural computed tomography algorithm implemented based on some embodiments of the disclosed technology when imaging during a complex change in topology.

FIG. 6 shows benefit of a signed distance function (SDF)-based object representation.

FIG. 7 shows a neural implicit representation of the SDF.

FIG. 8 shows an example of differentiable rendering of signed distance based neural implicit representation (NIR).

FIG. 9 shows a distance field representation algorithm (DiFiR-CT) based on some embodiments of the disclosed technology.

FIG. 10 shows intensity-only and spatially-aware object segmentation approaches used in DiFiR-CT.

FIG. 11 shows DiFiR-CT accurately depicts the position of a circle, despite high motion during acquisition.

FIGS. 12A and 12B show DiFiR-CT reconstruction of an object with angular displacement with varying spatial and temporal regularization.

FIG. 13 shows DiFiR-CT reconstruction with varying noise.

FIG. 14 shows DiFiR-CT-SegSI accurately depicts the moving dots with two attenuations and high angular displacements.

FIG. 15 shows DiFiR-CT improves single and multiple gantry rotation imaging of a “beating” circle.

FIG. 16 shows DiFiR-CT improves imaging of a complex change in topology.

FIG. 17 shows an example image reconstruction method based on some embodiments of the disclosed technology.

FIG. 18 shows another example image reconstruction method based on some embodiments of the disclosed technology.

FIG. 19 shows another example image reconstruction method based on some embodiments of the disclosed technology.

FIG. 20 shows another example image reconstruction method based on some embodiments of the disclosed technology.

### DETAILED DESCRIPTION

Section headings are used in the present document only for ease of understanding and do not limit scope of the embodiments to the section in which they are described.

Disclosed are methods, materials and devices that pertain to an image reconstruction that can improve the quality of medical imaging, such as computed tomography (CT) and magnetic resonance imaging (MRI).

The quality of medical images can be significantly impaired when objects move during imaging. As a result, to improve image quality, patients are often asked to lie still, body parts of interest may be held in place for particularly long scans, and pediatric patients may be sedated.

However, in certain scenarios, motion cannot be avoided. For example, when imaging the chest, both respiratory and cardiac motion are unavoidable and can impair image quality, so scanners aim to acquire data as quickly as possible. In addition, while estimating the underlying motion can be used to correct images, correctly solving for complex motions such as the motion of the heart or lungs is extremely challenging.

The disclosed technology can be implemented in various embodiments to improve imaging of moving objects which, among other features and benefits, overcome the above limitations. Specifically, some embodiments of the disclosed technology do not rely on motion estimation and can take advantage of data acquired during both stationary and moving periods. In some embodiments, an object-based approach is utilized to represent the position of object boundaries. Coupled with advances in neural networks, images with improved quality and movies which depict the motion that occurred can be obtained. This approach is complementary with fast imaging techniques and while demonstrated for CT imaging of the heart, can be applied to CT scenarios as well as other imaging modalities (such as MRI).

The disclosed technology can be implemented in some embodiments to correct errors in CT images created by the fact that objects move during data acquisition. Different from previous approaches that try to estimate or solve for the motion, an image reconstruction approach based on some embodiments of the disclosed technology is flexible enough to allow for complex and fast motions while at the same time, providing constraints that push the approach to favor solutions which match the acquired data and are realistic.

In some embodiments of the disclosed technology, an image reconstruction method represents a scene based on the location of object boundaries. The image reconstruction method does not use a matrix/grid to represent this information but an “implicit” approach via neural networks. The image reconstruction method parameterizes how these boundaries can evolve over space and time, and performs an optimization that aims to match the acquired data and a set of constraints that penalize unrealistic motions.

In some embodiments of the disclosed technology, the image reconstruction method may be applied to blurred features to clean them up.

In some embodiments of the disclosed technology, the image reconstruction method may be used to entirely replace current methods if significant motion is expected to limit even the initial image result.

While this patent document describes some embodiments of the disclosed technology in the context of correcting motion of heart structures despite fast scanner rotation speed, the disclosed technology can also be used in cases with longer acquisition times such as in CT scanning during radiation therapy and CT scanning with a C-arm device. These are additional clinical cases where motion can occur during data acquisition.

X-ray computed tomography (CT)'s ability to noninvasively and volumetrically evaluate patient anatomy with high spatial resolution has led to a widespread clinical use. Unfortunately, image quality can be reduced if object motion occurs during the acquisition. This is particularly concerning when imaging fast moving structures (e.g., the heart) as current single-source conebeam CT scanners require 200-300 ms to perform a full gantry rotation. As a result, while CT is used for the evaluation of suspected coronary artery disease and acute chest pain, even small motions (e.g., 1.2 mm) lead to significant blurring of key structures such as coronary arteries. Further, motion of highly-attenuating, metallic devices can further impair clinical assessment. In addition to static, single frame imaging, there is growing interest in using CT to evaluate heart dynamics. Various techniques have been developed to avoid, reduce, and/or correct motion artifacts. Broadly, these approaches aim to either reduce the amount of data needed for image reconstruction or correct for the underlying object motion.

The disclosed technology can be implemented in some embodiments to provide a novel neural network architecture for time-resolved reconstruction of CT images of moving objects without estimation or correction of object motion. Neural networks have proven useful in computer vision and medical imaging learning problems such as classification and segmentation. In addition, neural networks can serve an efficient framework for representation of complex scenes (i.e., neural representation). A key benefit of neural representations is that they are highly flexible, can represent arbitrarily complex scenes, and can be easily optimized via gradient descent.

Recently, neural representation has been combined with volumetric differentiable renderers to perform optimization directly from 2D images (projections). The CT image reconstruction problem is analogous to accurately solving the inverse volumetric object rendering problem dealt in previous works.

Therefore, a neural representation-based image reconstruction algorithm is capable of producing time-resolved images from conventional CT data (sinograms) and when applied to imaging data of moving objects, can reduce motion artifacts as compared with conventional reconstruction without the need for an explicit motion model.

In some embodiments, the neural representation-based image reconstruction algorithm can utilize a boundary-based representation and jointly estimate the position and displacement of object boundaries to facilitate optimization. Specifically, the neural representation-based image reconstruction algorithm implemented based on some embodiments can represent a target image as objects-of-interest with temporally-evolving boundaries instead of as scalar-valued intensity image or set of images, by using a signed distance function (SDF) as a spatiotemporal implicit representation. In some embodiments, the neural representation-based image reconstruction algorithm can perform an optimization to solve for the implicit representation which is most consistent with the acquired sinogram. Instead of using data-driven priors, the neural representation-based image reconstruction algorithm implemented based on some embodiments can promote convergence towards low frequency solutions which results in physically plausible reconstructions. This is achieved by using Fourier coefficients to represent the temporal evolution of the SDF. Therefore, the disclosed technology is not limited to any type of object motion nor require additional data (additional CT projections, motion field estimates, or physiologic signals such as the ECG). In this way, the neural representation-based image reconstruction algorithm implemented based on some embodiments provides a flexible method to accurately resolves motion of object boundaries.

In some embodiments, the neural representation-based image reconstruction algorithm implemented based on some embodiments utilizes CT image reconstruction as an object boundary-based problem and enables accurate reconstruction of moving objects.

In some embodiments, the neural representation-based image reconstruction algorithm can combine neural rendering with implicit representations to reconstruct objects undergoing fast and complex deformations.

In some embodiments, a neural computed tomography algorithm (NeuralCT) implemented based on some embodiments of the disclosed technology can reconstruct movies of moving objects imaged with standard axial CT acquisitions without assuming priors related to the motion. The neural computed tomography algorithm (NeuralCT) implemented based on some embodiments of the disclosed technology is robust to the choice of hyperparameters and can be readily used to solve for a wide range of motions.

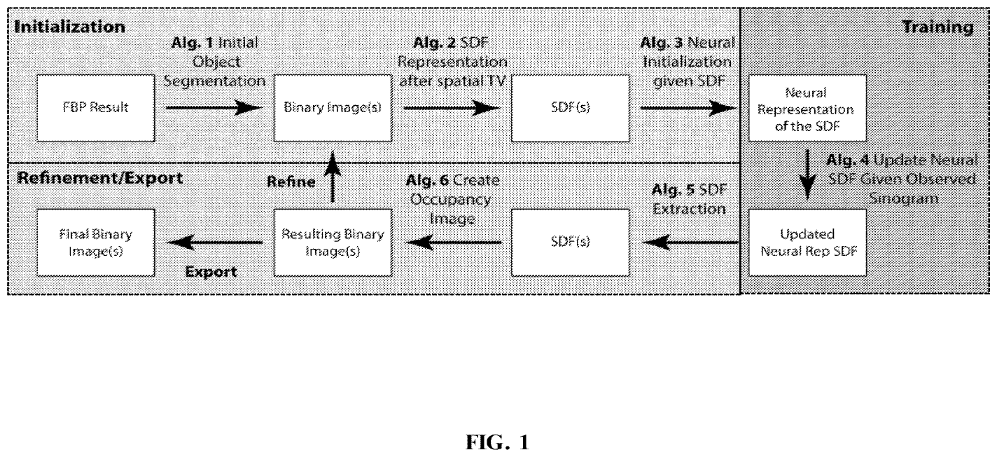

FIG. 1 shows an example of a neural computed tomography algorithm implemented based on some embodiments of the disclosed technology.

A neural computed tomography algorithm (NeuralCT) implemented based on some embodiments of the disclosed technology represents the spatiotemporal attenuation map of a moving object both explicitly in the image domain as well as implicitly in the neural domain and performs optimization in both of these domains across three stages: (1) initialization; (2) training; and (3) refinement/export. The initialization obtains well-behaved signed distance function (SDF) representations based on filtered back projection (FBP) reconstructed images using an intensity-based segmentation, encodes the resulting segmentation as signed distance functions, and performs the explicit-to-implicit representation conversion. The training aims to update neural SDF representation to match the sinogram data. The refinement/export stage includes the implicit-to-explicit conversion and creation of occupancy images. A neural computed tomography algorithm (NeuralCT) implemented based on some embodiments of the disclosed technology results improved when it is repeated using the results of the first prediction.

The design of the neural computed tomography algorithm (NeuralCT) implemented based on some embodiments of the disclosed technology leverages the fact that the spatiotemporal attenuation map of a moving object can be represented both explicitly in the image domain as well as implicitly in the neural domain, for example, as weights of a neural network. Thus, a neural computed tomography algorithm (NeuralCT) implemented based on some embodiments of the disclosed technology performs optimization in both of these domains across three stages: (1) initialization; (2) training; and (3) refinement/export, as outlined in FIG. 1.

In some implementations, the initialization is intended to obtain well behaved gradients (that satisfy eikonal constraint) instead of using a random initialization for the neural SDF representation. In some implementations, Algorithm 1 (e.g., Alg. 1 in FIG. 1) identifies objects of interest in an image reconstructed via filtered back projection (FBP) using an intensity-based segmentation. Algorithms 2 (e.g., Alg. 2 in FIG. 1) encodes the resulting segmentation (binary background-foreground) image as signed distance functions and Algorithm 3 (e.g., Alg. 3 in FIG. 1) performs the explicit-to-implicit representation conversion.

In some implementations, the training stage includes Algorithm 4 (e.g., Alg. 4 in FIG. 1) which is responsible for updating (training) the neural SDF representation to match the sinogram data.

In some implementations, the refinement/export includes Algorithm 5 (e.g., Alg. 5 in FIG. 1) which performs the implicit-to-explicit conversion and Algorithm 6 (e.g., Alg. 6 in FIG. 1) which creates the occupancy image for the discretized SDF map.

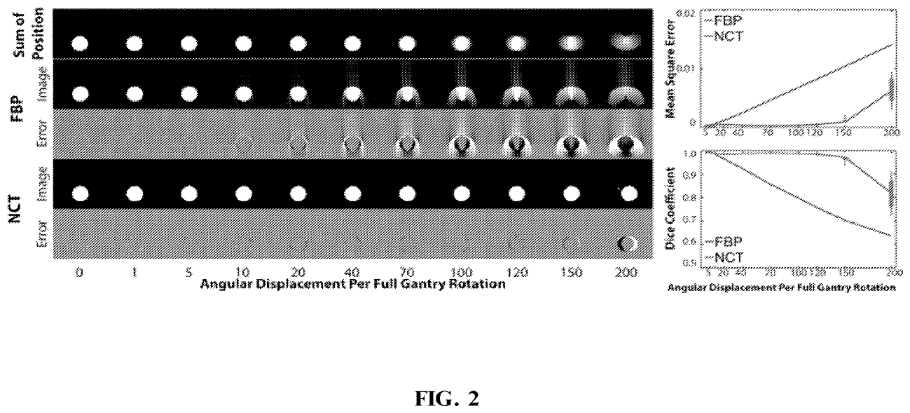

FIG. 2 shows filtered back projection (FBP) reconstructed images and images generated by a neural computed tomography algorithm implemented based on some embodiments of the disclosed technology.

Referring to FIG. 2, the neural computed tomography algorithm (NeuralCT) implemented based on some embodiments of the disclosed technology (NCT) accurately depicts the position of a circle, despite high angular motion during acquisition. In one example, FBP images are degraded with increasing motion during data acquisition. NCT improves delineation of the translating vessel, both visually and via Mean Squared Error and Dice Coefficient metrics. Error bars represent the standard deviation observed from 5 NCT results using different random initialization of the networks. All images are reconstructions using 360-degrees of projections. Displacement is imparted as an angular translation (in degrees) as indicated by the legend.

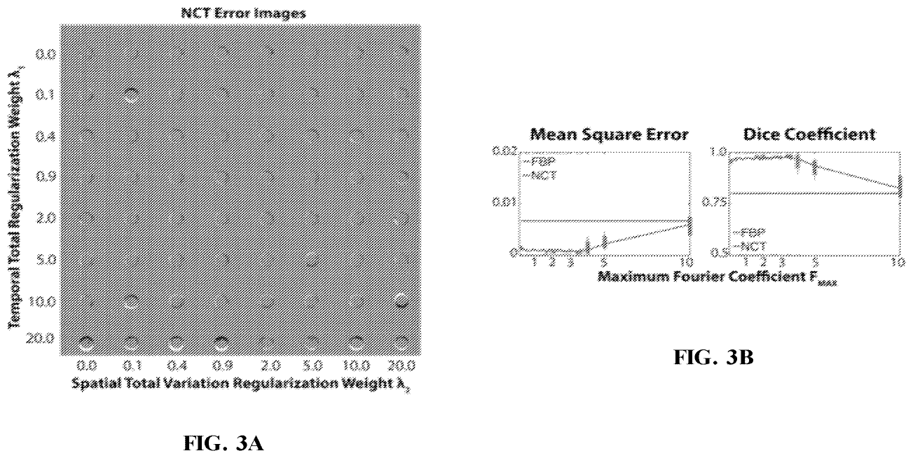

FIGS. 3A and 3B show an example of a neural computed tomography algorithm reconstruction of an object with angular displacement=100° with varying spatial and temporal regularization (FIG. 3A) and Fₘₐₓ (FIG. 3B). NCT yields accurate image reconstruction over a range of spatial TV regularization. However, NCT reconstruct can become inaccurate if temporal regularization is too highly penalized. This corresponds to making the solution more stationary. NCT results are also robust for a wide-range of Fₘₐₓ. At Fₘₐₓ>10, the increased parameterization can lead to overfitting but robust results can be achieved with lower Fₘₐₓ.

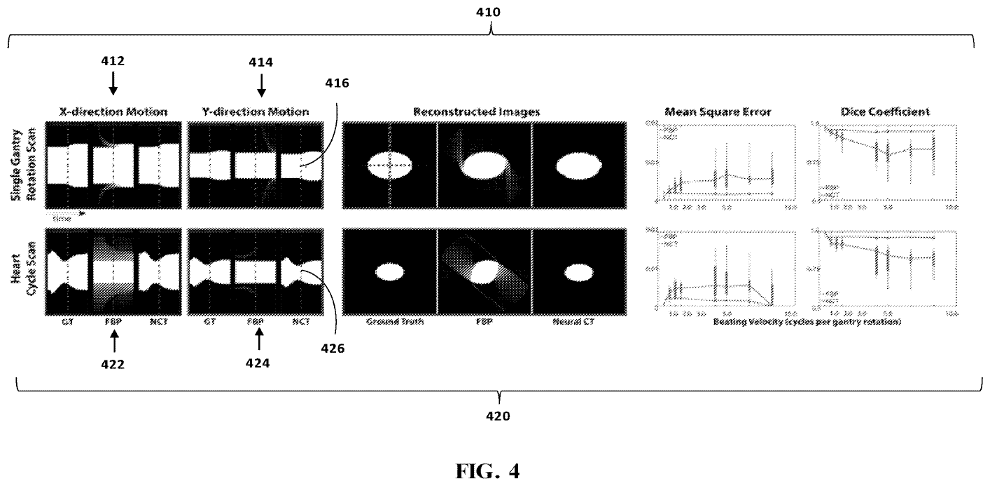

FIG. 4 shows an example of a neural computed tomography algorithm implemented based on some embodiments of the disclosed technology (NCT) that improves single (410) and multiple (420) gantry rotation imaging. In FIG. 4, 410 shows NCT improves image quality, relative to FBP for both cases. The motion of the X (412, 422) and Y (414, 424) axes is shown to illustrate how FBP blurring is improved by NCT. Images reconstructed at the middle of the data acquisition (416, 426) are shown in the middle panel. In FIG. 4, 420 shows NCT leverages the additional temporal information available when multiple gantry rotations are acquired and improves image quality, over FBP, without modification.

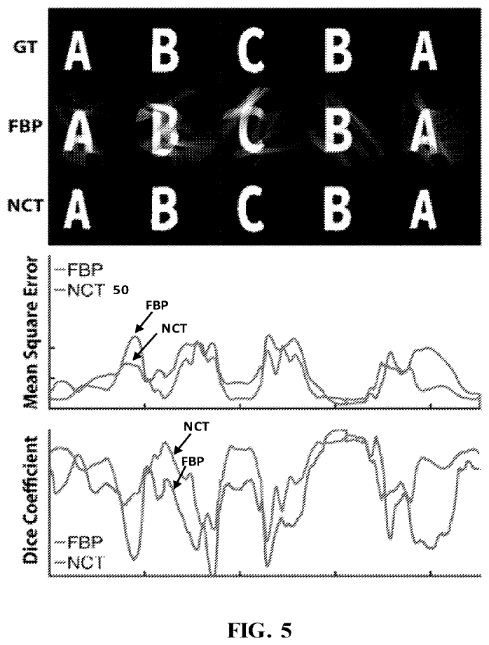

FIG. 5 shows evaluation of the neural computed tomography algorithm implemented based on some embodiments of the disclosed technology (NCT) when imaging during a complex change in topology. Without modification of the framework or tuning of parameters, NCT improved imaging of a complex scene. Further, the approach did so without estimation of motion or a prior information.

Motion during acquisition of a set of projections can lead to significant motion artifacts in computed tomography reconstructions despite fast acquisition of individual views. In cases such as cardiac imaging, motion may be unavoidable and evaluating motion may be of clinical interest. Reconstructing images with reduced motion artifacts have typically been achieved by developing systems with faster gantry rotation or using algorithms which measure and/or estimate the displacements. However, these approaches have had limited success due to both physical constraints as well as the challenge of estimating/measuring non-rigid, temporally varying, and patient-specific motions. The disclosed technology can be implemented in some embodiments to provide a novel reconstruction framework, a neural computed tomography algorithm (NeuralCT) implemented based on some embodiments of the disclosed technology, to generate time-resolved images free from motion artifacts. The disclosed technology can be implemented in some embodiments to utilize a neural implicit approach and does not require estimation or modeling of the underlying motion. Instead, boundaries are represented using a signed distance metric and neural implicit framework. The disclosed technology can be implemented in some embodiments to utilize ‘analysis-by-synthesis’ to identify a solution consistent with the acquired sinogram as well as spatial and temporal consistency constraints. We illustrate the utility of the neural computed tomography algorithm (NeuralCT) implemented based on some embodiments of the disclosed technology in three progressively more complex scenarios: translation of a small circle, heartbeat-like change in an ellipse's diameter, and complex topological deformation. Without hyperparameter tuning or change to the architecture, the neural computed tomography algorithm (NeuralCT) implemented based on some embodiments of the disclosed technology provides high quality image reconstruction for all three motions, as compared to filtered backprojection using mean-square-error and Dice metrics. Code and data will be publicly released upon acceptance.

### I. INTRODUCTION

X-ray computed tomography (CT)'s ability to noninvasively and volumetrically evaluate patient anatomy with high spatial resolution has led to a widespread clinical use. Unfortunately, image quality can be reduced if object motion occurs during the acquisition. This is particularly concerning when imaging fast moving structures (e.g., the heart) as current single-source conebeam CT scanners require 200-300 ms to perform a full gantry rotation. As a result, while CT is used for the evaluation of suspected coronary artery disease and acute chest pain, even small motions (˜1.2 mm) lead to significant blurring of key structures such as coronary arteries. Further, motion of highly-attenuating, metallic devices can further impair clinical assessment. In addition to static, single frame imaging, there is growing interest in using CT to evaluate heart dynamics. Various techniques have been developed to avoid, reduce, and/or correct motion artifacts. Broadly, these approaches aim to either reduce the amount of data needed for image reconstruction or correct for the underlying object motion. However, as described in Section II, current methods are limited.

The disclosed technology can be implemented in some embodiments to provide a new approach for time-resolved reconstruction of CT images of moving objects without estimation or correction of object motion. The disclosed technology can be implemented in some embodiments to provide a novel neural network architecture. Neural networks have proven useful in computer vision and medical imaging learning problems such as classification and segmentation. In addition, neural networks can serve an efficient framework for representation of complex scenes (i.e., neural representation). A key benefit of neural representations is that they are highly flexible, can represent arbitrarily complex scenes, and can be easily optimized via gradient descent.

Recently, neural representation has been combined with volumetric differentiable renderers to perform optimization directly from 2D images (projections). The CT image reconstruction problem is analogous to accurately solving the inverse volumetric object rendering problem dealt in previous works.

Therefore, the disclosed technology can be implemented in some embodiments to provide a neural representation-based image reconstruction algorithm capable of producing time-resolved images from conventional CT data (sinograms) and when applied to imaging data of moving objects, can reduce motion artifacts as compared with conventional reconstruction without the need for an explicit motion model.

The disclosed technology can be implemented in some embodiments to utilize a boundary-based representation and jointly estimate the position and displacement of object boundaries to facilitate optimization. Specifically, the disclosed technology can be implemented in some embodiments to represent a target image as objects-of-interest with temporally-evolving boundaries instead of as scalar-valued intensity image or set of images, e.g., by using the signed distance function (SDFs) as a spatiotemporal implicit representation. The optimization based on some embodiments of the disclosed technology then solves for the implicit representation which is most consistent with the acquired sinogram. Instead of using data-driven priors, the disclosed technology can be implemented in some embodiments to promote convergence towards low frequency solutions which results in physically plausible reconstructions. This is achieved by using Fourier coefficients to represent the temporal evolution of the SDF. Therefore, some embodiments of the disclosed technology do not assume any type of object motion nor require additional data (additional CT projections, motion field estimates, or physiologic signals such as the ECG). The disclosed technology can be implemented in some embodiments to provide a flexible method to accurately resolve motion of object boundaries.

In this patent document, we pose CT image reconstruction as an object boundary-based problem and show that this enables accurate reconstruction of moving objects. In addition, we present a pipeline, the neural computed tomography algorithm (NeuralCT), which combines neural rendering with implicit representations to reconstruct objects undergoing fast and complex deformations. Furthermore, the neural computed tomography algorithm (NeuralCT) implemented based on some embodiments of the disclosed technology can reconstruct movies of moving objects imaged with standard axial CT acquisitions without assuming priors related to the motion. We also highlight that the neural computed tomography algorithm (NeuralCT) implemented based on some embodiments of the disclosed technology is robust to the choice of hyperparameters and can be readily used to solve for a wide range of motions.

Section II below will discuss prior works related to the neural computed tomography algorithm (NeuralCT) implemented based on some embodiments of the disclosed technology. A component-wise description of the neural computed tomography algorithm (NeuralCT) implemented based on some embodiments of the disclosed technology is covered in Section III followed by implementation details in Section IV. Sections V and VI respectively detail the experiments and results.

### A. Avoidance of Motion Artifacts

Image artifacts can be minimized by limiting motion during the acquisition. When possible, anatomy of interest can be held still. When imaging the heart, cardiac motion can be reduced by imaging during portions of the cardiac cycle with relatively small motion (either the end-systole or diastasis periods). However, this can be limited in noncompliant patients or in the case of heart imaging, in patients with high heart rates. Further, static imaging precludes assessment of important motion such as joint articulation or cardiac dynamics.

Faster gantry rotation speeds can also be used to reduce motion artifacts. Increased rotation speed for single-source systems is mechanically challenging and requires an increase in x-ray tube power to maintain similar image quality. However, dual-source systems have been shown to improve the temporal resolution. Unfortunately, the increased mechanical complexity or additional sources has limited design of systems with more than two sources. Nonmechanical, electron beam CT systems, initially introduced in the 1980s, can achieve fast acquisition times (˜50 ms) but suboptimal image quality has limited routine clinical use. As a result, current single-source conebeam CT systems reconstruct images with temporal footprints>50 ms which can result in artifacts due to heart motion.

### B. Motion Estimation Approaches

Correcting motion artifacts by jointly estimating image intensity and motion over time on a per-pixel basis is significantly underdetermined. Therefore, estimating motion using computer vision approaches can prove beneficial. For example, partial angle images can be used to estimate and correct for temporally-constant but spatially-varying motion. One such approach has been developed into a clinical solution and been shown to improve image quality and reduce the presence of artifacts. However, validation of these methods has been limited especially when correcting complex motions. However, the development of advanced phantoms such as XCAT have recently enabled improved evaluation. In addition, recent work has leverage machine learning to improve motion estimation.

### C. Motion Artifact Removal

Machine learning has also been used to correct motion artifacts in reconstructed images. In some implementations, a deep convolutional neural network (CNN) to compensate for both rigid and nonrigid motion artifacts. In some implementations, three CNNs are used to reduce the metal artifacts created by pacemakers. The classification of coronary artery plaques blurred by motion has been improved by fine tuning of inception v3 CNN.

### D. Neural Implicit Representations and Reconstruction

There has been a large body of work exploring neural implicit representations (NIRs) for efficient storage of high resolution information. This is due to the fact that NIRs are continuously differentiable, which allows for theoretically infinite resolution and efficient optimization using classic gradient descent techniques. In contrast, conventional representations require significantly higher memory (voxels), make compromises on topology information (point clouds) or give unrealistic surfaces post optimization (meshes).

Classic shape reconstruction NIRs may be used for novel view synthesis and multi-view reconstruction. Recently, NIRs have been used to improve CT and MR imaging. In some implementations, NIRs can be used to solve inverse problem of CT reconstruction. Such techniques have since shown to enable super resolution and improve sparse CT reconstruction. Broadly, these approaches incorporate the data acquisition process to optimize the NIRs for static objects. In contrast, the focus of this paper is to reconstruct objects that move during data acquisition. While prior methods have extended NIRs to dynamic scenes, they seek to model the temporal change in intensity that is a step function for a moving object with uniform intensity. Our key insight is to instead represent object boundaries as SDFs, a physically motivated representation, to accurately capture boundary motion and enable time-resolved reconstruction. Moreover, we demonstrate that our technique can utilize the result of static reconstruction methods (in our case, filtered backprojection) as an initialization to reduce motion artifacts.

### III. FORMULATION OF NEURAL COMPUTED TOMOGRAPHY ALGORITHM (NEURALCT)

The disclosed technology can be implemented in some embodiments to provide two key components, (A) implicit representation and (B) differentiable rendering, before outlining the specific design of the neural computed tomography algorithm (NeuralCT) implemented based on some embodiments of the disclosed technology in Section IV.

### A. Implicit Representations and Inverse Problems

3D medical images are conventionally represented using volume elements (voxels), which are a direct extension of 2D picture elements (pixels). However, voxel-based representation of high resolution objects is computationally expensive. Meshes are an alternative which can represent the ‘surface’ information of a 3D object using a piece-wise triangulation. However, once a mesh is defined, topological issues can arise upon deformation.

As their name suggests, implicit representations seek to store information implicitly as a zero level set of a higher dimensional function. This can overcome the limitations associated with both voxel- and mesh-based representations. Specifically, the signed distance of a point in 3D space to a nearby surface can be used as a representation which avoids the topological issues associated with meshes as the signed distance map can be deformed without creating “holes” or “breaks” in the surface. Further, encoding the signed distance map with neural network enables a compressed representation of the object.

In some embodiments of the disclosed technology, a scene is implicitly represented using a vector of signed distance functions (SDF) ƒ: [symbol] ^{N}× [symbol] × [symbol] ^{K} to map the spatiotemporal coordinate [symbol] x,t [symbol] ∈ [symbol] ^{N}× [symbol] to the boundaries of K objects in spacetime represented by their SDF values ƒ(x,t)∈ [symbol] ^{K} where N is the number of spatial dimensions and K is the number of objects represented. The key benefit of using an SDF representation is that non-boundary locations provide information about the boundary displacement. This avoids having to explicitly track the boundary displacement, as is the case with other representations such as meshes or occupancy functions.

Numerically solving an implicit representation inverse problem requires a discrete representation. One conventional approach is to transform the object representation into a dense set of scalar values (e.g., a voxel grid). However, fitting a neural network to the representation is an efficient and differentiable alternative. Further, a neural network compactly adapts the spatiotemporal resolution of the representation to the complexity of the underlying object. In this work, we approximate the SDF representation ƒ(x,t) by a neural network ĝ(x,t;w), where w is a set of network parameters.

### B. Optimization Via Differentiable Rendering

In the absence of a known ground truth, ‘analysis-by-synthesis’ methods can be used to identify a solution that explains the observed data (in a different domain). Differentiable rendering is one such technique and is conventionally used to identify the shape Ŝ of an object that best ‘explains’ its acquired projection set {P₁,P₂, . . . P_{N}}. For a shape S, we achieve this by minimizing the error defined as the norm of differences between predicted and observed projections, i.e.

$$\hat{S} = {arg min}_{s} {\sum }_{i = 0}^{N} {‖ {P}_{i} - R ( s ; {θ}_{i} ) ‖}_{p} \qquad ( 1 )$$

Here R(S;θᵢ) is the rendering operator that projects a shape S from ‘shape space’ to ‘projection space’ based on CT gantry position θᵢ. To enable gradient backpropagation and optimizing via standard gradient descent methods, the rendering operator R should be differentiable.

### IV. Neural Computed Tomography Algorithm (NeuralCT)

In some embodiments the neural computed tomography algorithm (NeuralCT) may include the following core components.

### A. Architecture

As noted in Section III, we approximate the spatiotemporal SDF function ƒ(x,t) of an object via a neural network ĝ(x,t;w). We do this by creating two sub-networks that jointly approximate the stationary SDF ĝ_{E} as well as its spatiotemporal evolution ĝ_{V}.

EncoderNet: For each location x∈ [symbol] ^{N}, the stationary component of the object's SDF is represented using a network EncoderNet ĝ_{E}(x;w_{E}): [symbol] → [symbol] ^{K}.

VelocityNet: For each location x∈ [symbol] ^{N}, the temporal evolution of an object's SDF is parameterized using Fourier coefficients {A₀,A₁, . . . ,A_{MK},B₀,B₁, . . . ,B_{MK}} where (Aᵢ,Bᵢ) respectively are the coefficients for sines and cosines. The coefficients are fit by a network ĝ_{F}(x,t;w_{F}): [symbol] ^{N}→ [symbol] ^{2MK}. Then, ĝ_{V}(x,t;w_{F}) is be computed as:

$${\hat{g}}_{V} ( x , t ; {w}_{F} ) = \frac{1}{M} {\sum }_{i = 0}^{M} {A}_{i} ( x ) sin ( 2 {πω}_{i} t ) + {B}_{i} ( x ) cos ( 2 {πω}_{i} t ) \qquad ( 2 )$$

Here, ωᵢ˜N(0,Fₘₐₓ) is a random variable sampled from a normal distribution with standard deviation Fₘₐₓ. In practice, the maximum acceleration of natural objects will have physical constraints. This allows us to bandlimit the Fourier representation and parameterize the network with Fₘₐₓ. We determined Fₘₐₓ empirically with the goal of encouraging VelocityNet to represent more physically-relevant displacement fields.

EncoderNet and VelocityNet values are summed to estimate the SDF value of an object at every spatiotemporal position.

In some implementations of the disclosed technology, SIRENs, an efficient framework capable of capturing high frequency information, are used to implement both EncoderNet and VelocityNet. As a result, the overall representation is given by

$$\hat{g} ( x , t ; w ) = {\hat{g}}_{V} ( x , t ; {w}_{F} ) + {\hat{g}}_{E} ( x ; {w}_{E} ) \qquad ( 3 )$$

where w is the union of EncoderNet weights w_{E} and VelocityNet weights w_{F}, i.e.,

w=w_{E}∪w_{F}.

### B. Renderer

In CT imaging, the acquired sinogram r(l,θ) represents the attenuation accumulated by x-rays traversing from the source to a specific detector position l at a gantry position θ.

As described above, the gantry rotates over time t such that, if an object has a spatial, temporal attenuation intensity I(x,t), then the resultant sinogram value at detector position l is given by:

$$r ( l , θ ) = {\int }_{u} I ( x , t ) {Γ}_{θ} ( t ) du \qquad ( 4 )$$

where u is the path the ray traverses through the scene and Γ_{θ}(t)∈SO₂ is the time-varying rotation matrix which describes the gantry rotation by an angle θ about the center of the scene.

In some implementations, image reconstruction is desired on an N-dimensional grid of size d. Therefore, the neural implicit SDF estimate can be discretized to obtain \hat{ƒ}_{□}(x,t,k,ĝ). Occupancy of this discretized representation can be computed via ζ(x), defined as:

$$ζ ( x ) = min ( 1 , max ( 0 , μ * ( σ ( x ) - 0.5 ) ) ) \qquad ( 5 )$$

where μ is a scaling factor that controls the sharpness of the boundary and σ refers to the sigmoid function.

Then, using the attenuation intensity of the object a(k), we can estimate of the spatiotemporal intensity map Î(x,t) as

$$\hat{I} ( x , t ) = a ( k ) ζ ( {\hat{f}}_{} ( x , t , k , \hat{g} ) ) \qquad ( 6 )$$

The sinogram represents a discrete set of spatially- and angularly-integrated measurements in terms of both l and θ. Therefore, the disclosed technology can be implemented in some embodiments to perform both the forward rendering/projection operations at sub-pixel resolution to remove aliasing artifacts, for example, by upsampling the derived occupancy grid \hat{ƒ}(x,t;w), kₛₐₘₚ times using bilinear interpolation. Then, after calculating the projection using the above integral, r(l,θ) is downsampled via average pooling.

The steps outlined above result in a differentiable rendering operator R(·;θ): [symbol] ^{d}^{^{N}}× [symbol] → [symbol] ^{d}^{^{N-1}} which can map an SDF representation g(x,t;w) to the projection domain \hat{r}(l,θ) via appropriate discretization.

### V. How to Reconstruct Using Neural Computed Tomography Algorithm (NeuralCT)

The design of the neural computed tomography algorithm (NeuralCT) implemented based on some embodiments of the disclosed technology leverages the fact that the spatiotemporal attenuation map of a moving object can be represented both explicitly in the image domain as well as implicitly in the neural domain. That is, as weights of a neural network. Thus, the neural computed tomography algorithm (NeuralCT) implemented based on some embodiments of the disclosed technology performs optimization in both of these domains across three stages: Initialization, Training, Refinement/Export as outlined in FIG. 1.

Briefly, Initialization is intended to obtain well behaved gradients (that satisfy eikonal constraint) instead of using a random initialization for the neural SDF representation. To do so, Algorithm 1 identifies objects of interest in an image reconstructed via filtered back projection (FBP) using an intensity-based segmentation. Algorithm 2 encodes the resulting segmentation (binary background-foreground) image as signed distance functions and Algorithm 3 performs the explicit-to-implicit representation conversion.

The Training portion begins with Algorithm 4 which is responsible for updating (a.k.a. training) the neural SDF representation to match the sinogram data. Subsequently, Algorithm 5 performs the implicit-to-explicit conversion and Algorithm 6 creates the occupancy image for the discretized SDF map.

We observed improved results when the neural computed tomography algorithm (NeuralCT) is re-initialized using the result of its first prediction. Therefore, Refinement/Export consists of applying Algorithms 2-6 on the initial result of the neural computed tomography algorithm (NeuralCT).

### A. Initialization

The SDF of K foreground objects of interest is initialized using the filtered back projection (FBP) reconstruction images I_{FBP}(x,t). Here, the number of objects of interest K is defined apriori. Defining both a background class as well as several additional classes κ such that K′=K+κ+1 improved initialization.

Algorithm 1
Object Segmentation

| | |
|---|---|
| Input: | FBP images I_{FBP} (x, t)∀t ∈ T, |
| | number of objects K, |
| | buffer classes κ |
| Output: | Binary classification images C(x, t, k )∀t ∈ T |

| | |
|---|---|
| 1 | Randomly, select τ ⊂ T |
| 2 | Segmentor ← GMM (I_{FBP} (x, t), k′)t ∈ τ |
| 3 | G(x, t, k′) ← Segmentor.fit (I_{FBP} (x, t)∀t ∈ T) |
| 4 | M ← {···, Σ_{x∈Ω} G(x, t, cᵢ), ···} |
| 5 | ${c}_{background} ← {arg max}_{c} M$ |
| 6 | M ← M.delete (c_{background}) |
| 7 | {cᵢ} ← TopKIndices(M) |
| 8 | C(x, t, k) ← G(x, t, {cᵢ}) |
| 9 | return C(x, t, k); |

Algorithm 2
Convert Binary images to SDF

| | |
|---|---|
| Input: | Binary class images C(x,t,k)∀t ∈ T |
| Output: | SDF images \hat{f}(x,t,k)∀t ∈ T |

| | |
|---|---|
| 1 | \tilde{B}(x,t,k) ← TotalVariationMinimizer(C(x,t,k)) |
| 2 | \tilde{D}(x,t,k) ← DistanceTransform(\tilde{B}(x,t,k)) |
| 3 | \hat{f}(x,t,k) ← TotalVariationMinimizer(\tilde{D}(x,t,k)) |
| 4 | return \hat{f}(x,t,k); |

Algorithm 3
Initializing Neural SDF Representation

| | |
|---|---|
| Input: | Neural SDF g(x, t; w), |
| | SDF images \hat{f}(x, t, k) |
| Output: | Initialized neural SDF ĝₒ(x, t; w)∀t ∈ T |

| | |
|---|---|
| 1 | ĝ(x, t; w) = g (x, t; rand(w)) |
| 2 | for iteration < maxIterations do |
| 3 | \| Sample t ~ T |
| 4 | $\vert {L}_{SDF} ← \frac{1}{\vert Ω \vert} {\sum }_{x ∈ Ω} \vert \hat{g} ( x , t ; w ) - \hat{f} ( x , t ) \vert$ |
| 5 | $\vert {L}_{Eikonal} ← \frac{1}{\vert Ω \vert} {\sum }_{x ∈ Ω} \vert {‖ {∇}_{x} \hat{g} ( x , t ; w ) ‖}_{2} - 1 \vert$ |
| 6 | \| L ← L_{SDF} + λL_{Eikonal} |
| 7 | \| w ← w − α∇L |
| 8 | end |
| 9 | ĝₒ(x, t; w) ← ĝ(x, t; w) |
| 10 | return ĝₒ(x, t; w); |

Algorithm 1 performs intensity-based segmentation using a Gaussian Mixture Model GMM (I_{FBP}(x,t),k′) to create a segmentation image G(x,t). This results in binary classification images C(x,t,k′). From these binary images, the mass M(k′) of each foreground class is calculated. After removal of the background c_{background}, defined as the class with the largest mass, the binary images C(x,t,k) for the K largest classes are kept.

Binary class images C(x,t,k) representing pixels of each label across time are then converted into SDF images \hat{ƒ}(x,t,k) using Algorithm 2. In some embodiments, total variation minimization is used to smooth the images and then performs the distance transform over the entire domain to obtain SDF images \hat{ƒ}(x,t,k).

To create neural implicit representations from the explicit SDF images \hat{ƒ}(x,t,k), the disclosed technology can be implemented in some embodiments to perform optimization as described in Algorithm 3. A randomly initialized neural network g(x,t;w) is optimized into a network ĝₒ(x,t;w) which best approximates the explicit SDF image ĝ(x,t;w)≈\hat{ƒ}(x,t,k). The optimization is directly supervised and aims to minimize the SDF differences L_{SDF} and satisfy the Eikonal constraint L_{Eikonal}. The optimization ends when the maximum number of iterations maxIterations have been reached.

### B. Training

Algorithm 4
Optimizing Neural SDF Representation

| | |
|---|---|
| Input: | Initialized SDF ĝₒ(x, t; w)∀t ∈ T, acquired |
| | sinogram r(l, θ) |
| Output: | Optimized SDF ĝ(x, t; w)∀t ∈ T |

| | |
|---|---|
| 1 | for iteration < maxIterations do |
| 2 | \| Sample t ~ T; |
| 3 | \| Compute projection \hat{r}(l, θ) ← R(Î(x, t), θ(t)); |
| 4 | $\vert {L}_{Sinogram} ← \frac{1}{\vert Ω \vert} {\sum }_{x ∈ Ω} \vert r ( l , θ ) - \hat{r} ( l , θ ) \vert$ |
| 5 | $\vert {L}_{Eikonal} ← \frac{1}{\vert Ω \vert} {\sum }_{x ∈ Ω} \vert {‖ {∇}_{x} {\hat{g}}_{o} ( x , t ; w ) ‖}_{2} - 1 \vert$ |
| 6 | $\vert {L}_{TVS} ← \frac{1}{\vert Ω \vert} {\sum }_{x ∈ Ω} {‖ {∇}_{x} {\hat{g}}_{o} ( x , t ; w ) ‖}_{1}$ |
| 7 | $\vert {L}_{TVT} ← \frac{1}{\vert Ω \vert} {\sum }_{x ∈ Ω} {‖ {∇}_{t} {\hat{g}}_{o} ( x , t ; w ) ‖}_{1}$ |
| 8 | \| L ← L_{Sinogram} + λ₁L_{Eikonal} + λ₂L_{TVS} + λ₃L_{TVT} |
| 9 | \| w ← w − α∇L |
| 10 | \| if L_{Sinogram} < minLoss then |
| 11 | \| \| break |
| 12 | \| end |
| 13 | end |
| 14 | ĝ(x, t; w) ← ĝₒ(x, t; w); |
| 15 | return ĝ(x, t; w); |

At this point, the SDF representation ĝₒ(x,t;w) includes motion artifacts present in I_{FBP}. Algorithm 4 optimizes the neural network SDF representation ĝ(x,t;w) to best explain the acquired sinogram r(l,θ) with the minimum total variation in space ∥∇ₓĝ(x,t;w)∥₁, and time ∥∇ₜĝ(x,t;w)∥₁,

The current neural implicit SDF prediction ĝ(x,t;w) can be converted to a spatiotemporal intensity map Î(x,t) and projected to the sinogram domain via previously described renderer (Section IV) R(I(x,t),θ). This results in a sinogram estimate \hat{r}(l,θ) used to calculate the sinogram loss L_{Sinogram}—the difference between the current estimate and the acquired sinogram r(l,θ). This loss is combined with the Eikonal constraint L_{Eikonal} and spatial L_{TVs} and temporal TV L_{TVT} loss terms to improve optimization. The optimization is performed until the projection loss L_{Sinogram} decreased below a certain threshold minLoss or maximum number of iterations maxIterations is reached.

### C. Refinement/Exporting

To generate spatiotemporal intensity images from the neural SDF representation, we first convert the neural SDF into a set of explicit SDF images \hat{ƒ}_{□}(x,t,k). This is achieved by sampling the neural SDF ĝ(x,t;w) over a n-dimensional grid at a desired spatial resolution (Algorithm 5). The resulting SDF image is then binarized \hat{B}(x,t,k)=ζ(\hat{ƒ}_{□}(x,t,k)) (Algorithm 6). Binarized images \hat{B}(x,t,k;w) are then used for Refinement, a second pass through the algorithm (starting with Algorithm 2). After Refinement, images are exported and analyzed.

### VI. EXPERIMENTS

### A. Implementation Details

The image resolution used during training is set to n=128 with upsampling factor kₛₐₘₚ=2. For optimal performance, we used Fₘₐₓ=3.0 and μ=50. In Algorithm 1, we sampled τ such that |τ|=0.02*|T| and set κ=3. For Algorithms 3 & 4 the learning rate is α=1×10⁻⁵ and decayed by a factor of 0.95 every 200 steps. The optimization procedures is run with maxIterations=5000, minLoss=0.08, and λ=0.1. For best results, we trained with minibatch |t|=20. As described below, we observed optimal results with λ₁=0.1, λ₂=0.5, and λ₃=0.5. All experiments are performed using a 2D parallel beam CT geometry of a single foreground class (i.e., K=1). Optimizing the neural computed tomography algorithm (NeuralCT) implemented based on some embodiments of the disclosed technology takes ˜30 mins on a single NVIDIA 1080Ti GPU.

### B. Evaluation of Neural Computed Tomography Algorithm (NeuralCT)

First, we simulated a simple cause of motion corruption: imaging a bright circle undergoing translation. This is intended to mimic motion of a coronary artery. We used this toy problem to explore the impact of λ₁, λ₂, and Fₘₐₓ on the ability of the neural computed tomography algorithm (NeuralCT) implemented based on some embodiments of the disclosed technology to faithfully reconstruct a moving object. λ₁ and λ₂ are the weight of spatial and temporal total variation in the cost function used to supervise learning and Fₘₐₓ is the maximum Fourier coefficient available for representation of the temporal changes.

The impact of these parameters on the neural computed tomography algorithm (NeuralCT) implemented based on some embodiments of the disclosed technology is evaluated via parameter sweeps. First, λ₁ and λ₂ are varied (0, 0.05, 0.1, 0.15, 0.3, 0.5, 0.9, 1.5, 3.0, 5.0, 10.0) with Fₘₐₓ set to 3.0. Then, Fₘₐₓ is varied from 0 to 10.0 with λ₁=0.5 and λ₂=0.5.

In some implementations, a range of angular displacements per gantry rotation (0, 1, 5, 10, 20, 40, 70, 100, 120, 150, and 200 degrees per gantry rotation) is evaluated. The neural computed tomography algorithm (NeuralCT) results are compared to both the ground-truth vessel image as well as the FBP result using the metrics described below.

### C. Imaging of Nonrigid, Heart-Like Motion

To evaluate the utility of the neural computed tomography algorithm (NeuralCT) implemented based on some embodiments of the disclosed technology during non-rigid motion, we modeled heart-like motion using an ellipse with time-varying axes. In addition to static imaging, cine CT imaging can be used to evaluate heart size and dynamics. Therefore, we evaluated two imaging approaches: 1) Single-gantry rotation imaging where a sinogram spanning 360 degrees is acquired during which the ellipse diameter changes. 2) Full heart cycle imaging where multiple (typically 4-5) gantry rotations are obtained spanning the entire period of the heart-like motion. The neural computed tomography algorithm (NeuralCT) implemented based on some embodiments of the disclosed technology readily incorporate multiple rotation data into the reconstruction framework without modification. In this scenario, the neural computed tomography algorithm (NeuralCT) implemented based on some embodiments of the disclosed technology results are compared to FBP reconstructions centered at the same temporal position.

### D. Imaging of Complex Deformations

Lastly, we demonstrate the ability of the neural computed tomography algorithm (NeuralCT) implemented based on some embodiments of the disclosed technology to reconstruct scenes with complex topological change. To do so, we created a complex scene where the letter “A” transforms into “B”, then into “C”, then back to “B” and “A”. Without changes to the neural computed tomography algorithm (NeuralCT) parameters (spatial/temporal weighting and Fourier coefficients), the neural computed tomography algorithm (NeuralCT) results are compared to FBP imaging.

### E. Metrics to Evaluate Image Quality

The neural computed tomography algorithm (NeuralCT) implemented based on some embodiments of the disclosed technology is compared to conventional FBP images using mean-square error (MSE) and the foreground's Dice coefficient where FBP images are thresholded using half of the foreground intensity. We did not compare the neural computed tomography algorithm (NeuralCT) to motion correction methods. This is motivated by the fact that the improvement obtained via correction depends on the suitability of the motion model in the correction scheme to the motion present in the acquired data. Given that current correction methods have been designed for clinical scenarios, our motion scenarios are not expected to represent realistic use cases. For the neural computed tomography algorithm (NeuralCT), the average of 5 independent optimizations, each initialized with a different random seed are discussed in this patent document.

### VII. RESULTS

### A. Moving Coronary Vessel Imaging

As shown by the images and metrics in FIG. 2, the neural computed tomography algorithm (NeuralCT) implemented based on some embodiments of the disclosed technology accurately reconstructed images of a small circle while FBP images showed significant artifacts (λ₁=0.5, λ₂=0.5, and Fₘₐₓ=3.0). Low MSE and high (>0.9) Dice is maintained for angular displacements of up to 150° during data acquisition. FIG. 2 also illustrates how the neural computed tomography algorithm (NeuralCT) is impacted by displacements >150°. While the shape of the circle is fairly preserved, increased MSE and decreased Dice occur due to reconstruction of the circle at the incorrect temporal position.

### B. Evaluation of Neural Computed Tomography Algorithm (NeuralCT)

The neural computed tomography algorithm (NeuralCT) reconstruction of a circle with translation=100° per gantry rotation is used to evaluate the robustness of NCT to changes in λ₁, λ₂, and Fₘₐₓ. FIG. 3A illustrates the minor impact of changing the strength of spatial (λ₁) and temporal (λ₂) regularization while maintaining Fₘₐₓ=3.0. Accurate reconstruction is achieved over a wide range of regularization strength. FIG. 3B illustrates the performance of the neural computed tomography algorithm (NeuralCT) with λ₁=0.5 and λ₂=0.5 when varying Fₘₐₓ. Decreasing Fₘₐₓ limits the temporal evolution that can be represented by the neural implicit approach. However, we observed robust neural computed tomography algorithm (NeuralCT) reconstruction performance even at low Fₘₐₓ values. Reconstruction accuracy decreased at high Fₘₐₓ. This suggests that introduction of high frequency coefficients can result in overfitting.

### C. Imaging Nonrigid, Heart-Like Motion with Neural Computed Tomography Algorithm (NeuralCT)

Without modification of the parameters identified above (λ₁=0.5, λ₂=0.5, and Fₘₐₓ=3.0), NCT successfully reconstructed images of the heart-like motion for both single gantry and full heart cycle imaging. Relative to FBP, the neural computed tomography algorithm (NeuralCT) implemented based on some embodiments of the disclosed technology can improve imaging metrics when imaging an ellipse with changing dimensions. FIG. 4 illustrates these differences. The change in axes dimension is shown by the left column (temporal reformats along the x- and y-direction). Metrics of image quality (MSE and Dice) are shown on the right. For full heart cycle imaging with beating velocity=3.0, NCT decreased MSE (FBP: median 0.005, IQR 0.003-0.009, NCT: median 0.001, IQR 0.001-0.002). Further, NCT increased the percentage of frames with MSE<0.005 from 46% (FBP) to 100%. NCT also improved Dice (FBP: median 0.84, IQR 0.73-0.88 to NCT: median 0.96, IQR 0.95-0.97).

### D. Imaging Complex Topology Changes with Neural Computed Tomography Algorithm (NeuralCT)

Without modification of the neural computed tomography algorithm (NeuralCT) framework or parameters (λ₁=0.5, λ₂=0.5, and Fₘₐₓ=3.0), NCT successfully reconstructed data acquired during a complex letter warping scene. FIG. 5 shows the stationary periods of the scene with both the groundtruth (top) as well as FBP (middle) and the neural computed tomography algorithm (NeuralCT) (bottom) reconstructions. The neural computed tomography algorithm (NeuralCT) implemented based on some embodiments of the disclosed technology may significantly reduce the severity of artifacts observed with FBP. The plot of MSE and Dice scores as a function of time further illustrate the improvement. NCT decreased error as measured via MSE (FBP: median 0.011, IR 0.008-0.019 to NCT: median 0.007, IQR 0.004-0.013). Further, NCT increased the percentage of the frames with MSE<0.005 from 15.2% to 34%. DICE scores also improved with NCT (FBP: median 0.78, IQR 0.66-0.85, NCT: median 0.86, IQR 0.80-0.91). The percentage of frames with Dice >0.85 increased from 25.8% for FBP to 58.8% with NCT.

The neural computed tomography algorithm (NeuralCT) implemented based on some embodiments of the disclosed technology successfully combines implicit representation of an object by a signed distance function (SDF) with differentiable rendering to enable time-resolved imaging free from motion artifacts despite data acquisition occurring during motion. Neural computed tomography algorithm (NeuralCT) implemented based on some embodiments of the disclosed technology takes advantage of several important features of SDFs-namely, that they represent movement of a boundary as a spatially and temporally smooth evolution. The neural computed tomography algorithm (NeuralCT) implemented based on some embodiments of the disclosed technology represents the scene as SDFs which evolve over time using an implicit representation; in this case, a neural network. Differentiable rendering is used to improve the estimate of the scene by comparing the observed CT data with the SDF-based representation in the sinogram domain. The framework also enables additional regularization such as penalizing deviations from the Eikonal constraint and minimizing spatial and temporal variations. We demonstrate the utility of the neural computed tomography algorithm (NeuralCT) in three different imaging scenarios without changes to the architecture. Specifically, the neural computed tomography algorithm (NeuralCT) implemented based on some embodiments of the disclosed technology is readily applied to objects with different motions as well as data spanning one or more than one gantry rotation. These cases highlight how Neural computed tomography algorithm (NeuralCT) implemented based on some embodiments of the disclosed technology can be used to accurately reconstruct objects undergoing 1) translation, 2) heartbeat-like affine changes in diameter, and 3) complex topological changes (warping of letters). This flexibility is facilitated by the fact that the neural computed tomography algorithm (NeuralCT) implemented based on some embodiments of the disclosed technology does not utilize an explicit motion model.

The neural computed tomography algorithm (NeuralCT) implemented based on some embodiments of the disclosed technology may provide an accurate reconstruction even with a fairly limited set of Fourier coefficients. This is likely due to the beneficial fact that SDFs used to parameterize the reconstruction problem evolve smoothly over space and time. We also observed reconstruction performance decreased if Fₘₐₓ increased. This highlights the benefit of limiting the complexity of the solution via Fₘₐₓ. An added advantage is that this hyperparameter has a simple physical interpretation, the bandwidth of the motion, which could facilitate its selection when applying the neural computed tomography algorithm (NeuralCT) to novel applications. Further, in our examples, a single foreground object is imaged with an empty background. However, in practice, the background class can be used to represent the position and intensity of static objects.

In some implementations, the neural computed tomography algorithm (NeuralCT) results are obtained without modification of parameters λ₁, λ₂, and Fₘₐₓ. However, the optimal choice of these parameters is expected to vary depending on the specific imaging scenarios. Further, the rotation speed of CT systems can depend on both the design of the system and the clinical protocol. Evaluating the optimal combination of parameters is left for future work where specific scenarios are evaluated.

In some implementations, the neural computed tomography algorithm (NeuralCT) implemented based on some embodiments of the disclosed technology can seamlessly reconstruct data acquired using two common CT acquisition approaches (single and multiple gantry rotation imaging). Specifically, the neural computed tomography algorithm (NeuralCT) implemented based on some embodiments of the disclosed technology may reconstruct imaging data spanning multiple gantry rotations simultaneously without explicit labeling of timepoints or the need for specific, complementary images. This is significantly different than approaches such as FBP which reconstruct each frame independently. As a result, in addition to improving single gantry reconstruction, the neural computed tomography algorithm (NeuralCT) may also improve reconstruction quality of each frame acquired during a multiple gantry acquisition by leveraging additional temporal information. While acquiring additional temporal information (beyond the typical half- or single gantry rotation) increases the dose delivered to the patient, the neural computed tomography algorithm (NeuralCT) may resolve the dynamics of objects that have been significantly hampered by imaging artifacts.

In some implementations, the neural computed tomography algorithm (NeuralCT) image quality is quantified relative to the ground truth and conventional FBP reconstruction instead of direct comparison to previously-reported motion-correction approaches. In some implementations, the neural computed tomography algorithm (NeuralCT) is not compared to techniques specially crafted for certain imaging scenarios such as translation of a coronary artery, because the performance of motion correction approaches to depend significantly on the specifics of the application. For example, motion-correction approaches to accurately reconstruct scenes when the object motion agrees with the method's motion model. However, limited performance may occur if the object motion is different from the motion model. While some embodiments do not require an a priori motion model, it is difficult to ensure that the examples adhere to the constraints incorporated into current approaches. Comparison to current motion-correction algorithms is planned for future work in specific clinical scenarios. In addition, as indicated above, the use of the neural computed tomography algorithm (NeuralCT) can be demonstrated using a single foreground class and imaging with a single-source 2D parallel beam geometry. This is done to simplify interpretability of our findings. While dual-source systems, fan- or cone-beam geometry, and multiple object scenes will change the relationship between object motion and acquired CT data, the neural computed tomography algorithm (NeuralCT) implemented based on some embodiments of the disclosed technology may improve image quality in these scenarios.

As discussed above, a novel reconstruction framework, the neural computed tomography algorithm (NeuralCT), can be used to reconstruct CT imaging data acquired during object motion in a time-resolved manner free from motion artifacts. The neural computed tomography algorithm implemented based on some embodiments leverages a neural implicit scheme and does not require a prior motion models or explicit motion estimation. Representing moving boundaries using a signed distance metric and neural implicit framework enables ‘analysis-by-synthesis’ to identify a solution consistent with the observed sinogram as well as spatial and temporal consistency constraints.

### Distance Field Representation to Resolve Motion Artifacts in Computed Tomography (DiFiR-CT)

Motion during data acquisition leads to artifacts in computed tomography (CT) reconstructions. In cases such as cardiac imaging, not only is motion unavoidable, but evaluating the motion of the object is of clinical interest. Reducing motion artifacts has typically been achieved by developing systems with faster gantry rotation or via algorithms which measure and/or estimate the displacement. However, these approaches have had limited success due to both physical constraints as well as the challenge of estimating non-rigid, temporally varying, and patient-specific motion fields.

The disclosed technology can be implemented in some embodiments to provide a novel reconstruction method which generates time-resolved, artifact-free images without estimation or explicit modeling of the motion.

In some embodiment, an analysis-by-synthesis approach is used to progressively regress a solution consistent with the acquired sinogram. In our method, we focus on the movement of object boundaries. Not only are the boundaries the source of image artifacts, but object boundaries can simultaneously be used to represent both the object as well as its motion over time without need for an explicit motion model. We represent the object boundaries via a signed distance function (SDF) which can be efficiently modeled using neural networks. As a result, optimization can be performed under spatial and temporal smoothness constraints without the need for explicit motion estimation.

The utility of DiFiR-CT in three imaging scenarios with increasing motion complexity will be discussed: translation of a small circle, heart-like change in an ellipse's diameter, and a complex topological deformation. Compared to filtered backprojection, DiFiR-CT provides high quality image reconstruction for all three motions without hyperparameter tuning or change to the architecture. In some embodiment, DiFiR-CT's robustness to noise in the acquired sinogram reveals that its reconstruction is accurate across a wide range of noise levels. In some embodiments, DiFiR-CT may be used for multi-intensity scenes, which shows the importance of the initial segmentation providing a realistic initialization.

In some embodiment, projection data can be used to accurately estimate a temporally-evolving scene without the need for explicit motion estimation using a neural implicit representation and analysis-by-synthesis approach.

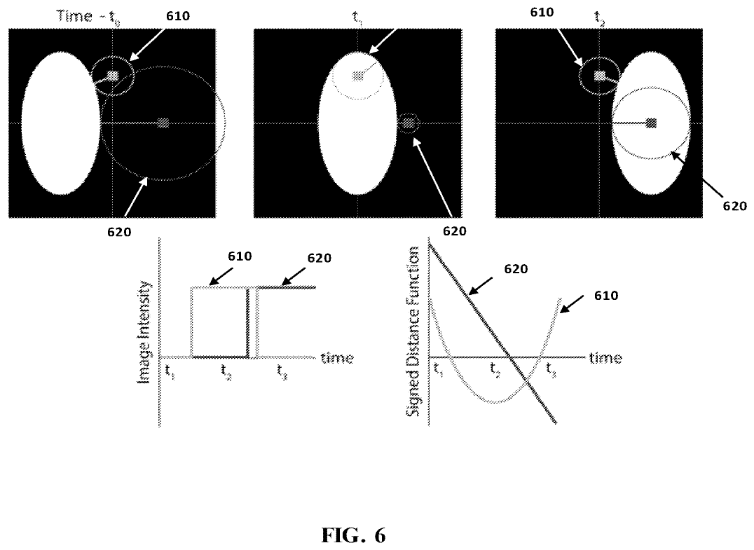

FIG. 6 shows benefit of a signed distance function (SDF)-based object representation. The SDF of a moving object boundary is a smooth function of time. In this scene, a white ellipse moves from left to right over a black background, across 3 frames. The per-pixel image intensity (observed at 620 and 610 locations) changes over time in a step-like fashion. However, the signed distance value changes smoothly which makes it amenable for fitting a Lipschitz continuous function.

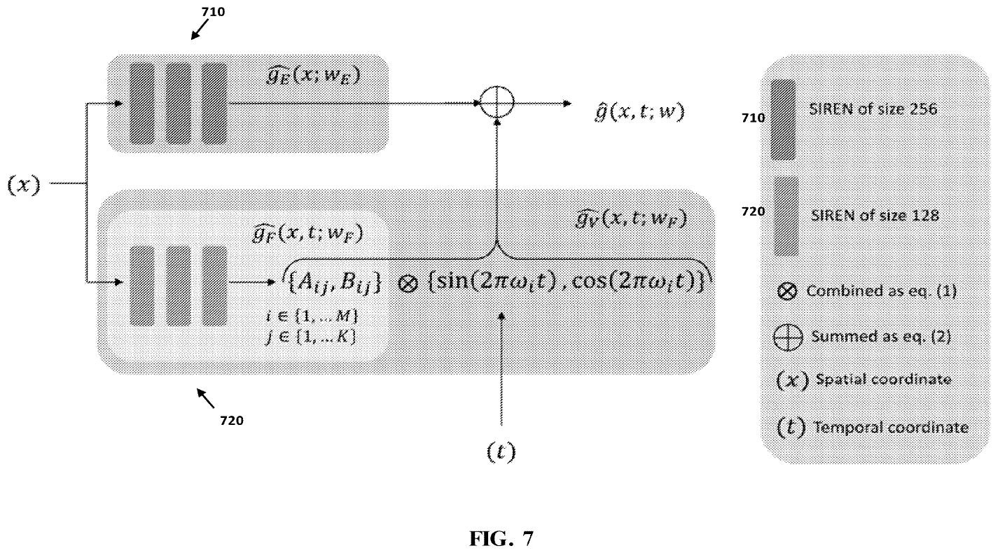

FIG. 7 shows a neural implicit representation of the SDF. The SDF is approximated using a neural network ĝ(x,t;w) comprising two sub-networks which estimate the shape of the SDF g, as well as its spatiotemporal displacement ĝ_{V}. Neural networks are implemented as SIREN MLPs.

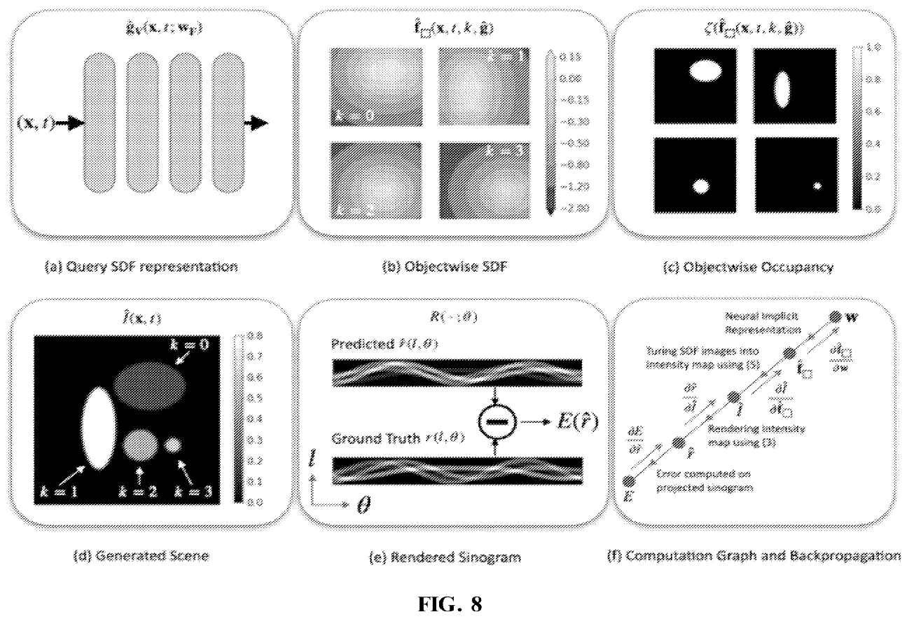

FIG. 8 shows an example of differentiable rendering of signed distance based NIR: (a) Given the object-wise spatiotemporal SDF representation ĝ(x,t;w), we query the network at each point x on the N-dimensional grid of size d at some time frame t. (b) This results in an object-wise discrete signed distance function for each of the k objects. (c) and (d) SDFs are then converted into object-wise occupancy values and subsequently combined to form the final scene by taking the dot product with the attenuation intensity a(k). In this illustration, a(0)=0.2, a(1)=0.8, a(2)=0.5 and a(3)=0.6. (e) This scene is rendered using R that results in a sinogram \hat{r} which can then be compared with ground truth sinogram r giving rise to an error functional E. (f) The entire computation graph is shown in which highlights that gradient from the functional E can be backpropagated through the renderer to the network weights w. This is crucial for optimizing the SDF representation ĝ(x,t;w) using only the projected sinogram data.

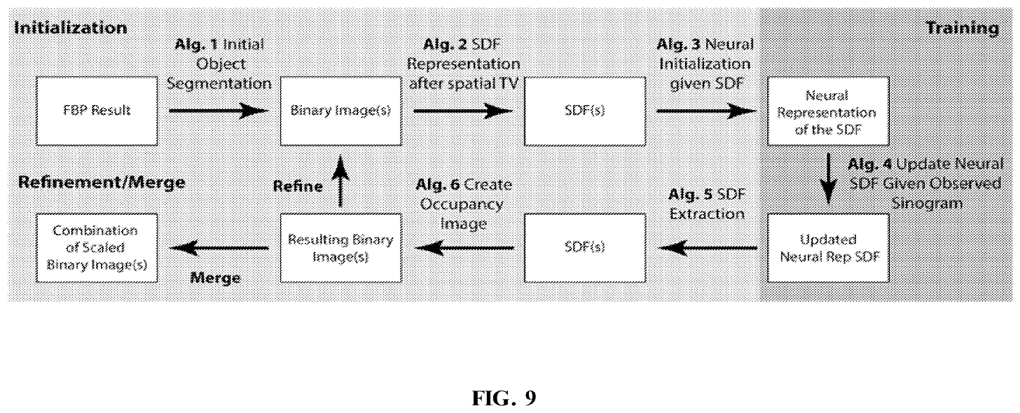

FIG. 9 shows a distance field representation algorithm (DiFiR-CT) based on some embodiments of the disclosed technology. DiFiR-CT represents the spatiotemporal attenuation map of a moving object both explicitly in the image domain as well as implicitly in the neural domain and performs optimization in both of these domains across three stages: Initialization, Training, Refinement/Export. Initialization obtains well-behaved SDF representations based on filtered back projection (FBP) reconstructed images using an intensity-based segmentation, encodes the resulting segmentation as signed distance functions, and performs the explicit-to-implicit representation conversion. Training aims to update neural SDF representation to match the sinogram data. Refinement/Merge includes the implicit-to-explicit conversion, creation of occupancy images, and scaling and combination of binary images. DiFiR-CT results improved when it is repeated using the results of the first prediction.

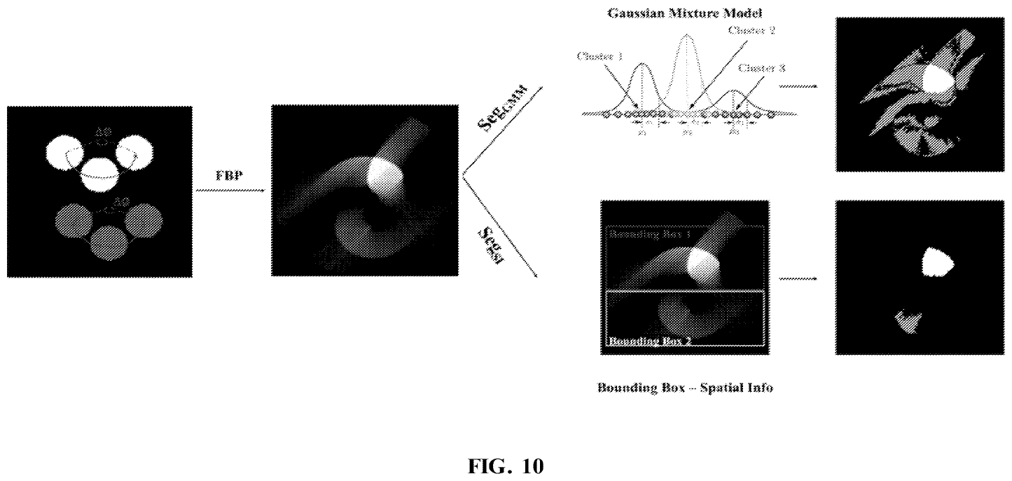

FIG. 10 shows intensity-only and spatially-aware object segmentation approaches used in DiFiR-CT. The first image shows the ground truth motion of two dots (top intensity=0.7, moving from left to right, bottom intensity=0.2, moving from right to left). Δθ is the angular displacement per gantry rotation. In FIG. 10, “SegGMM” indicates Gaussian mixture model incorrectly assigned the motion artifacts and the bottom dot as the same class, and “SegSI” indicates spatially aware segmentation utilized both spatial info (by setting bounding box in this example) and intensity info (thresholding) and led to correct detection of both top and bottom dots.

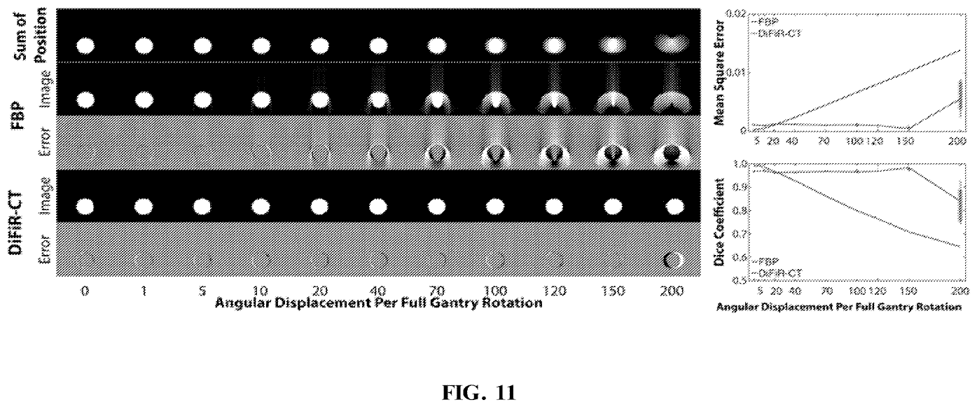

FIG. 11 shows DiFiR-CT accurately depicts the position of a circle, despite high motion during acquisition. FBP images are degraded with increasing motion during data acquisition. DiFiR-CT improves delineation of the translating circle. Error bars represent the standard deviation observed from 5 DiFiR-CT results using different random initialization of the networks. All images are reconstructions using 360-degrees of projections. Displacement is imparted as an angular translation (in degrees) as indicated by the legend.

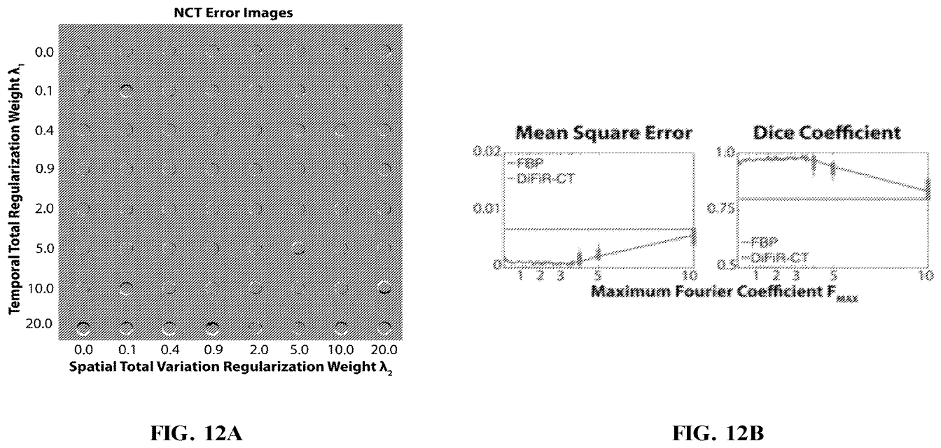

FIGS. 12A and 12B show DiFiR-CT reconstruction of an object with angular displacement=100° with varying spatial and temporal regularization (FIG. 12A) and Fₘₐₓ (FIG. 12B). DiFiR-CT yields accurate image reconstruction over a range of spatial TV regularization. However, DiFiR-CT reconstruct can become inaccurate if temporal regularization is too highly penalized (e.g., λ₁=20). This corresponds to making the solution more stationary. DiFiR-CT results are also robust for a wide-range of Fₘₐₓ. At Fₘₐₓ>10, the increased parameterization can lead to overfitting and robust results can be achieved with lower Fₘₐₓ.

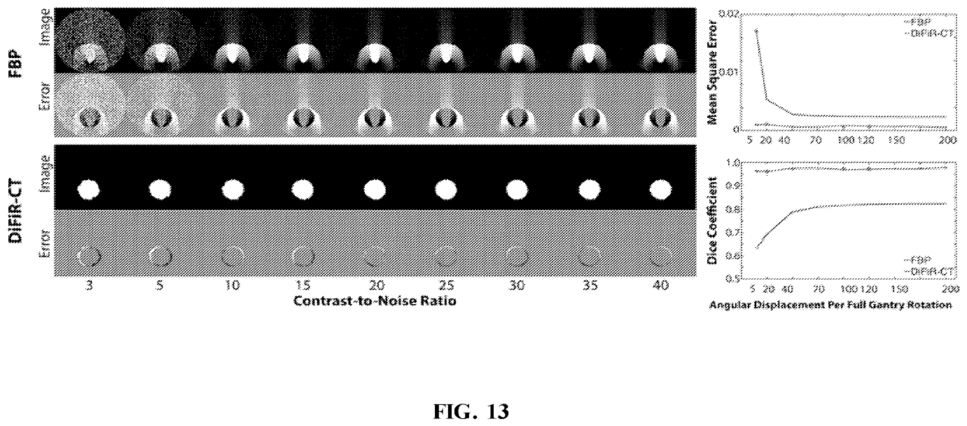

FIG. 13 shows DiFiR-CT reconstruction with varying noise. DiFiR-CT yields accurate image reconstruction over a range of contrast-to-noise ratios for a circle with angular displacement=100°. However, FBP shows a decrease in image reconstruction accuracy as the noise increase (CNR decreases). Median values of five noise realizations are shown for both FBP and DiFiR-CT results.

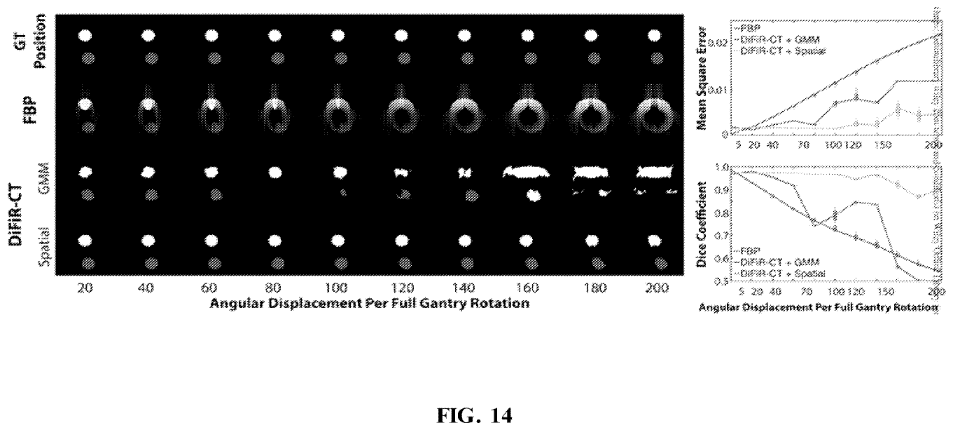

FIG. 14 shows DiFiR-CT-SegSI accurately depicts the moving dots with two attenuations and high angular displacements. DiFiR-CT-SegGMM failed to accurately reconstruct two circles when Δθ>60. DiFiR-CT-SegSI maintained high-quality motion-corrected reconstruction for all Δθ with higher DICE and lower MSE when compared with FBP and DiFiR-CT-GMM. Δθ indicates angular displacement per gantry rotation. FBP suffers from motion artifacts for all Δθ.

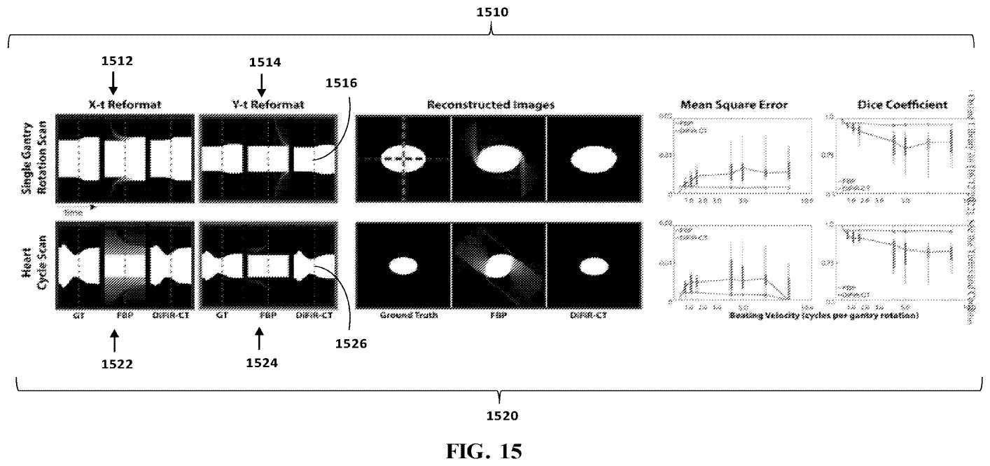

FIG. 15 shows DiFiR-CT improves single (1510) and multiple (1520) gantry rotation imaging of a “beating” circle. In FIG. 15, 1510 shows DiFiR-CT improves image quality, relative to FBP for both cases. The change in diameter of the circle along the x (1512, 1522) and y (1514, 1524) axes are shown to illustrate how FBP blurring is improved by DiFiR-CT. Images reconstructed at the middle of the data acquisition (1516, 1526) are shown in the middle panel. DiFiR-CT reduces the noticeable motion artifacts along the edges. In FIG. 15, 1520 shows DiFiR-CT can readily leverage additional temporal information available when multiple gantry rotations are acquired and improves image quality, over FBP, without modification.

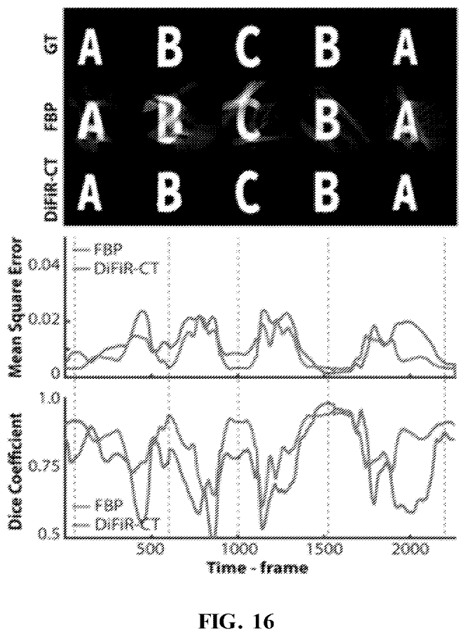

FIG. 16 shows DiFiR-CT improves imaging of a complex change in topology. Without modification of the framework or tuning of parameters, DiFiR-CT improved imaging of a complex scene. Further, the approach did so without estimation of motion or a prior information. The temporal location of the 5 frames is shown on the MSE and Dice plots as vertical dotted lines.

### I. Introduction

X-ray computed tomography (CT) can non-invasively and volumetrically evaluate patient anatomy with high spatial resolution, which has led to a widespread clinical use. Unfortunately, image quality can be reduced if motion occurs during the acquisition, despite fast acquisition of individual projections. This is particularly problematic when imaging fast moving structures (e.g., the heart) as current single-source conebeam scanners require 200-300 ms to perform a full gantry rotation. As a result, while CT is used for the evaluation of suspected coronary artery disease and acute chest pain, even small motions (˜1.2 mm) can result in significant blurring of key structures, such as coronary arteries. Further, motion of highly-attenuating structures, such as metallic devices, can further impair clinical assessment. Besides static, single frame imaging, CT is increasingly being used to evaluate heart dynamics. Various techniques have been developed to avoid, reduce, and/or correct motion artifacts. Broadly, approaches aim to either reduce the amount of data needed for image reconstruction or correct for the underlying object motion. However, as described in Section II., current methods remain limited.

The disclosed technology can be implemented in some embodiments to provide a new approach for time-resolved reconstruction of CT images of moving objects without estimation or explicit correction of the underlying object motion. Traditional intensity-based image reconstruction aims to represent each spatial position of the scene and therefore becomes suboptimal for scenes with motion as the problem becomes ill-posed. Our approach is based on the insight that object boundaries can simultaneously be used to represent an object's shape as well as its motion over time. As shown in the example in FIG. 6, a signed distance function-based representation provides an alternative, efficient representation to intensity mapping which we leverage to solve for both the shape and motion.

Referring to FIG. 6, consider a white 2D ellipse which moves from left to right, over a black background, across Y time frames. We can represent the scene at each time frame by measuring a per-pixel quantity ƒ(x) where x refers to the coordinate of a certain pixel in the image. Traditionally, ƒ(x) is the image intensity. In this example, ƒ(x) would assume a discrete value (white or black). Alternatively, ƒ(x) can be defined as a signed distance function, i.e., SDF (x) which is a continuous value equal to the smallest signed distance of x to the object's boundary (shown by the radii of green and blue circles). Note that SDF (x)<0 if x is inside the object's boundary and SDF (x)>0 when outside the object. While both definitions of ƒ(x) represent the object's state in each of the three frames, the change in ƒ(x) as a function of time (bottom) is different. The intensity-based representation (bottom left) leads to discontinuous changes over time which are hard to represent by a Lipschitz continuous function. On the other hand, the signed distance function (bottom right) yields a smooth curve which can be easily fitted. This example highlights how the signed distance may be a superior representation of an object's spatiotemporal state compared to the commonly used image intensity.

A discrete voxel grid can be used to represent the SDF of a scene but it leads to a huge memory footprint when extended to the temporal domain. This motivates our use of neural network-based representations, commonly referred to as neural implicit representations (NIRs), as they are capable of efficiently representing complex scenes. Neural representations are highly flexible, can represent arbitrarily complex scenes with only a few MB of memory, and can be easily optimized via gradient descent.

In some embodiments of the disclosed technology, NIRs can be used to map each spatiotemporal position to its corresponding signed distance. Each scene is represented as objects-of-interest with temporally-evolving boundaries by means of neural implicit SDF representation instead of as scalar-valued intensity image or set of images. Via optimization, we estimate the position and displacement of object boundaries over time which is most consistent with the acquired sinogram. Instead of using data-driven priors, we promote convergence towards low frequency solutions (i.e., physically plausible reconstructions) by using Fourier coefficients to represent the temporal evolution of the SDF and user-specified spatial and temporal smoothness constraints. Therefore, the disclosed technology can be implemented in some embodiments to solve for the objects position and displacement without assuming a particular type of object motion. It also does not require additional data (e.g., extra CT projections, motion field estimates, or physiologic signals such as the ECG).

In some implementations, projections can be used to identify the position of object boundaries. In addition, the disclosed technology can be implemented in some embodiments to address the challenges encountered with a moving scene. Further, in some implementations, NIRs can be optimized directly from projection images via volumetric differentiable renderers. Reconstruction of CT images is analogous to the inverse volumetric object rendering problem. In some embodiments, DiFiR-CT includes an algorithm that optimizes the spatiotemporal representation of a moving object in both the image domain and neural domain across three stages: Initialization (Section IV.A), Training (Section IV.B) and Refinement/Export (Section IV.C). As such, the disclosed technology can be implemented in some embodiments to provide a framework that is robust to the choice of hyperparameters and performs well despite the presence of noise in the acquired sinogram (Section V.) across several different problems (Section VI.).

In this patent document, we pose CT image reconstruction as an object boundary-based problem and show that this enables improved representation and reconstruction of moving objects. In addition, we present a pipeline, DiFiR-CT, which combines neural rendering with an implicit representation to reconstruct objects undergoing fast and complex deformations. Furthermore, DiFiR-CT may reconstruct movies of moving objects imaged with standard axial CT acquisitions without assuming priors related to the motion. In some embodiments, DiFiR-CT performance is robust to the choice of hyperparameters, is robust to noise in the acquired sinogram, and can be readily used to solve for a wide range of motions.

Section II below will discuss prior works related to DiFiR-CT. A component-wise description of DiFiR-CT is covered in Section III. followed by implementation details in Section IV. Sections V. and VI. respectively detail the experiments and results.

### II. BACKGROUND

### II.A. Avoidance, Estimation, or Reduction of Motion Artifacts

Image artifacts can be minimized by limiting motion during acquisition of CT data. While anatomy of interest can often be held still, this is challenging when imaging the heart due to its near-constant motion. Instead, artifacts due to cardiac motion can be reduced by imaging during portions of the cardiac cycle with relatively small motion (either the end-systole or diastasis periods). However, this can be of limited use in patients with high or irregular heart rates. Further, avoiding periods of object motion may limit assessment of key properties such as joint articulation or cardiac dynamics.

Faster gantry rotation can also be used to reduce motion artifacts. Increasing the rotation speed for single-source systems is mechanically challenging and requires an increase in x-ray tube power to maintain similar image quality. Dual-source systems have been shown to improve the temporal resolution. Unfortunately, the increased mechanical complexity or additional sources has limited design of systems with more than two sources. While non-mechanical, electron beam CT systems, initially introduced in the 1980s, can achieve fast acquisition times (˜50 ms), suboptimal image quality has limited clinical adoption. As a result, most current clinical CT systems are of the single-source design and reconstruct images with temporal footprints >100 ms which can result in significant motion artifacts when imaging cardiac structures.

Computer vision approaches have proven beneficial in solving the significantly underdetermined problem of jointly estimating image intensity and motion over time on a per-pixel basis. For example, partial angle images can be used to estimate and correct for temporally-constant but spatially-varying motion. One such approach (“SnapShot Freeze”, GE Healthcare) has been developed into a clinical solution and been shown to improve image quality and reduce the presence of artifacts. More recent work has leveraged machine learning to improve motion estimation. However, the ability of these methods correct complex motions remains unclear despite advanced phantoms such as XCAT being used to improve evaluation.

Machine learning has also been used to correct motion artifacts in reconstructed images. In some implementations, a deep convolutional neural network (CNN) to compensate for both rigid and nonrigid motion artifacts. Similarly, three CNNs may be used to reduce the metal artifacts created by pacemakers. The classification of coronary artery plaques blurred by motion has also been improved by fine tuning of inception v3 CNN.

### II.B. Neural Implicit Representations and Reconstruction

Neural implicit representations (NIRs) are an efficient approach for storage of high resolution information. This can be attributed to the fact that NIRs are continuously differentiable, which allows for both theoretically infinite resolution and efficient optimization using classic gradient descent techniques. In contrast, conventional representations either require significantly higher memory usage (voxels), make compromises regarding topology information (point clouds) or give unrealistic surfaces after optimization (meshes).

Since classic shape reconstruction works, NIRs have been used for novel view synthesis and multi-view reconstruction. Recently, NIRs have been used to improve CT and MR imaging. The use of NIRs to help solve inverse problem of CT reconstruction is first shown by Sun. Such techniques have since shown to enable super resolution and improve sparse CT reconstruction. In most cases, these approaches model the data acquisition process to optimize the NIRs for static objects. In contrast, the focus of this paper is to reconstruct objects that move during data acquisition. NIRs have been extended to dynamic scenes by warping a learned template scene with estimated motion field. In some embodiments of the disclosed technology, the scene and motion fields are modeled separately. Due to the reliance on a template, this method may fail to handle complex topology changes. However, the disclosed technology can be implemented in some embodiments to accurately capture boundary motion and enable time-resolved reconstruction by representing the position and motion of object boundaries via SDFs, a physically motivated representation. Moreover, the disclosed technology can be implemented in some embodiments to utilize the result of conventional reconstructions (e.g., filtered back-projection) as an effective initialization.

### III. DiFiR-CT

In some embodiments, the core components of DiFiR-CT may include the signed distance-based neural implicit representation and our differentiable renderer which allows for optimization of the representation via analysis-by-synthesis.

### III.A. Signed Distance-based NIR

A signed distance-based implicit representation seeks to store the object's shape as a zero level-set of a higher dimensional function. This can be extended to scenes with multiple objects by representing each object with their own signed distance function. In this work, we implicitly represent a scene using a vector of signed distance functions (SDF) ƒ: [symbol] _{N}× [symbol] → [symbol] ^{K} to map the spatiotemporal coordinate [symbol] x,t [symbol] ∈ [symbol] ^{N}× [symbol] to the boundaries of K objects in spacetime represented by their SDF values ƒ(x,t)∈ [symbol] ^{K} where N is the number of spatial dimensions and K is the number of objects represented. As discussed in Section II.D, fitting a neural network is an efficient and differentiable alternative to defining SDFs on a discrete set of grids. Further, a neural network compactly adapts the spatiotemporal resolution of the representation to the complexity of the underlying object.

We approximate the SDF representation ƒ(x,t) by a neural network ĝ(x,t;w), where w is the set of network weights (see FIG. 7). We do this by creating two sub-networks that jointly approximate the shape of the SDF ĝ_{E} as well as its spatiotemporal displacement ĝ_{V}. While this is done explicitly via two sub-networks and is intended to ensure the network had sufficient expressive capacity, optimization of the MLP for specific tasks is left for future work.

EncoderNet: For each location x∈ [symbol] ^{N}, the stationary component of the object's SDF is represented using a network EncoderNet ĝ_{E}(x;w_{E}): [symbol] → [symbol] ^{K}.

VelocityNet: For each location x∈ [symbol] ^{N}, the temporal evolution of an object's SDF is parameterized using Fourier coefficients (Aᵢⱼ,Bᵢⱼ), i∈{1, . . . , M}, j∈{1, . . . ,K} which are respectively the coefficients for sines and cosines of K objects. The coefficients are fit by a network ĝ_{F}(x,t;w_{F}): [symbol] ^{N}→ [symbol] ^{2MK}. Then, ĝ_{V}(x,t;w_{F}): [symbol] ^{N}→ [symbol] ^{K} is computed as:

$${\hat{g}}_{V} ( x , t ; {w}_{F} ) = \qquad ( 7 )$$

$$\frac{1}{M} [ \begin{matrix}{A}_{11} ( x ) & … & {A}_{1 M} ( x ) & {B}_{11} ( x ) & … & {B}_{1 M} ( x ) \\ {A}_{21} ( x ) & … & {A}_{2 M} ( x ) & {B}_{21} ( x ) & … & {B}_{2 M} ( x ) \\ ⋮ & ⋮ & ⋮ & ⋮ & ⋮ & ⋮ \\ {A}_{K 1} ( x ) & … & {A}_{KM} ( x ) & {B}_{K 1} ( x ) & … & {B}_{KM} ( x )\end{matrix} ] [ \begin{matrix}sin ( 2 {πω}_{1} t ) \\ sin ( 2 {πω}_{2} t ) \\ ⋮ \\ sin ( 2 {πω}_{M} t ) \\ cos ( 2 {πω}_{1} t ) \\ cos ( 2 {πω}_{2} t ) \\ ⋮ \\ cos ( 2 {πω}_{M} t )\end{matrix} ]$$

Here, ωᵢ˜N(0,Fₘₐₓ) is a random variable sampled from a normal distribution with standard deviation Fₘₐₓ (Hz). In some embodiments, sampling from a normal distribution (instead of a uniform distribution) led to better optimization. The random sampling is performed once, at the beginning of each reconstruction. In practice, we expect physical constraints allow for bandlimiting of the Fourier representation and parameterization of the network with Fₘₐₓ. We determined Fₘₐₓ empirically with the goal of encouraging VelocityNet to represent physically-relevant displacement fields and found M=128 frequencies to be sufficient for our test cases despite earlier results utilizing M=256. As shown in Equation 7, ĝ_{V}(x,t;w_{F}) yields a distance value based on the Fourier representation encoded by ĝ_{F}(x,t;w_{F}). In other words, ĝ_{V}(x,t;w_{F}) recombines the 2 MK output of ĝ_{F}(x,t;w_{F}) into the desired K-dimensional SDF displacement values such that they can be summed with the result from the EncoderNet.

In some embodiments of the disclosed technology, SIRENs are an efficient framework capable of capturing high frequency information and, therefore, are used to implement both EncoderNet and VelocityNet. As a result, the overall representation is given by

$$\hat{g} ( x , t ; w ) = {\hat{g}}_{V} ( x , t ; {w}_{F} ) + {\hat{g}}_{E} ( x ; {w}_{E} ) \qquad ( 8 )$$

where w is the union of EncoderNet weights w_{E} and VelocityNet weights w_{F}, i.e.,

w=w_{E}∪w_{F}.

### III.B. Differentiable Renderer

The differentiable renderer projects a spatiotemporal attenuation intensity I(x,t) to the sinogram domain r(l,θ) using a model of the CT system. This is done by integrating attenuation encountered by rays traveling from the source to a specific detector position/at a gantry position θ. As the gantry rotates over time t, the spatiotemporal attenuation intensity I(x,t) is rendered resulting in the sinogram value r(l,θ) as defined by the following integral:

$$r ( l , θ ) = {\int }_{u} I ( x , t ) Γ ( θ ( t ) ) du \qquad ( 9 )$$

where u is the path the ray traverses through the scene and Γ(θ(t)) is the time-varying two-dimensional rotation matrix which describes the gantry rotation by an angle θ about the center of the scene which varies as a function of time. Note that the computation for r(l,θ) can be performed differentiably using appropriate sampling of the above integral. This means that the spatiotemporal attenuation intensity I(x,t) can be updated based on some error functional E(r) defined on the rendered sinogram r(l,θ) by backpropagating its gradient ∂E(r)/∂r(l,θ).

In some embodiments of the disclosed technology, the attenuation intensity I(x,t) is encoded by means of object-wise spatiotemporal SDFs encoded in the network ĝ(x,t;w). Note, hat notation \hat{·} is used to distinguish quantities predicted by a neural network from ground truth values. Î(x,t) is extracted from ĝ(x,t;w) in a differentiable manner so that gradients of the error functional ∂E can be used to directly update the weights w of our implicit representation ĝ(x,t;w). As shown in FIG. 8, the process begins by uniformly querying the SDF representation ĝ(x,t;w) over the N-dimensional grid of size d at some time frame t. This results in a unique, albeit discrete, SDF representation \hat{ƒ}_{□}(x,t,k,ĝ) for each of the k objects as separate channels of the network's output volume. Note, the □ indicates a discrete representation so \hat{ƒ}_{□}(x,t,k,ĝ) is the estimate of the SDF sampled on a discrete pixel/voxel grid. This discrete representation is then converted into an occupancy via ζ(y), defined as:

$$ζ ( y ) = min ( 1 , max ( 0 , μ * ( σ ( y ) - 0.5 ) ) ) \qquad ( 10 )$$

where μ is a scaling factor that controls the sharpness of the boundary and σ refers to the sigmoid function. The spatiotemporal intensity map Î(x,t) can be computed by taking the inner product of per-channel occupancy and attenuation intensity a(k) (assumed to be known apriori).

$$\hat{I} ( x , t ) = a ( k ) ζ ( {\hat{f}}_{} ( x , t , k , \hat{g} ) ) \qquad ( 11 )$$

In order to perform accurate rendering and model the spatially-integrating detectors used for CT, the derived occupancy grid \hat{ƒ}(x,t;w), is upsampled kₛₐₘₚ times using bilinear interpolation before performing the rendering operation. Subsequently, the projected sinogram r(l,θ) is downsampled via average pooling. These steps describe a differentiable rendering operator R(·;θ): [symbol] ^{d}^{^{N}}× [symbol] → [symbol] ^{d}^{^{N-1}} which can map an SDF representation ĝ(x,t;w) to the projection domain \hat{r}(l,θ) via appropriate discretization. This procedure is end-to-end differentiable and allows for backpropagating gradients of a functional E such as the L₁ distance between r(l,θ) and \hat{r}(l,θ) to the network weights w.

### IV. RECONSTRUCTION USING DIFIR-CT

DiFiR-CT leverages the fact that the spatiotemporal attenuation map of a moving object can be represented both explicitly in the image domain as well as implicitly in the neural domain. That is, as weights of a neural network. As outlined in FIG. 9, DiFiR-CT performs optimization in both of these domains across three stages: Initialization, Training, Refinement/Export.

In some embodiments of the disclosed technology, Initialization is intended to obtain well-behaved gradients. One way we do this is by leveraging the fact that the norm of the gradient of the signed distance field is always unity (i.e., the Eikonal constraint). Further, instead of using a random initialization for the neural SDF representation, we utilize the filtered back projection result. Algorithm 1 identifies objects of interest in an image reconstructed via filtered back projection (FBP) using a simple intensity-based segmentation. Algorithm 2 encodes the resulting segmentation (binary background-foreground) image as signed distance functions and Algorithm 3 performs the explicit-to-implicit representation conversion.

The training portion begins with Algorithm 4 which is responsible for updating (a.k.a. training) the neural SDF representation to match the sinogram data. Subsequently, Algorithm 5 performs the implicit-to-explicit conversion and Algorithm 6 creates the occupancy image for the discretized SDF map.

Results improved when DiFiR-CT is re-initialized using the result of its first prediction. Therefore, Refinement/Export consists of applying Algorithms 2-6 on the initial result of DiFiR-CT.

### IV.A. Initialization

The SDF of K foreground objects of interest is initialized using the filtered back projection (FBP) reconstruction images I_{FBP}(x,t). Here, the number of objects of interest K is defined apriori. Defining both a background class as well as several additional classes κ such that K′=K+κ+1 improved initialization.

Algorithm 1 performs intensity-based segmentation using a Gaussian Mixture Model GMM (I_{FBP}(x,t),k′) to create segmentation images G(x,t). This results in binary classification images C(x,t,k′). From these binary images, the mass M(k′) of each foreground class is calculated. After removal of the background c_{background}, defined as the class with the largest mass, the binary images C(x,t,k) for the K largest classes are kept.

Algorithm 1

| | |
|---|---|
| Input: | FBP images I_{FBP}(x,t)∀t ∈ T |
| | number of objects K |
| | buffer classes κ |
| Output: | Binary classification images C(x,t,k)∀t ∈ T |

| | |
|---|---|
| 1 | Randomly, select τ ⊂ T |
| 2 | Segmentor ← GMM(I_{FBP}(x,t),k′)t ∈ τ |
| 3 | G(x,t,k′) ← Segmentor.fit(I_{FBP}(x,t)∀t ∈ T |
| 4 | M ←{•••,Σ_{x∈Ω}G(x,t,cᵢ),•••} |
| 5 | c_{background} ← [symbol] M |
| 6 | M ← M.delete(c_{background}) |
| 7 | {cᵢ} ← TopKIndices(M) |
| 8 | C(x,t,k) ← G(x,t,{cᵢ}) |
| 9 | return C(x,t,k); |

Algorithm 2
Convert Binary images to SDF

| | |
|---|---|
| Input: | Binary class images C(x,t,k)∀t ∈ T |
| Output: | SDF images \hat{f}(x,t,k)∀t ∈ T |

| | |
|---|---|
| 1 | \tilde{B}(x,t,k) ← TotalVariationMinimizer(C(x,t,k)) |
| 2 | \tilde{D}(x,t,k) ← DistanceTransform(\tilde{B}(x,t,k)) |
| 3 | \hat{f}(x,t,k) ← TotalVariationMinimizer(\tilde{D}(x,t,k)) |
| 4 | return \hat{f}(x,t,k); |

Binary class images C(x,t,k) representing pixels of each label across time are then converted into SDF images \hat{ƒ}(x,t,k) using Algorithm 2. Our approach uses total variation minimization to smooth the images and then performs the distance transform over the entire domain to obtain SDF images \hat{ƒ}(x,k).

To create neural implicit representations from the explicit SDF images \hat{ƒ}(x,t,k), the disclosed technology can be implemented in some embodiments to perform optimization as described in Algorithm 3. A randomly initialized neural network g(x,t;w) is optimized into a network ĝₒ(x,t;w) which best approximates the explicit SDF image ĝ(x,t;w)≈\hat{ƒ}(x,t,k) The optimization is directly supervised and aims to minimize the SDF differences L_{SDF} and satisfy the Eikonal constraint L_{Eikonal}. The optimization ends when the maximum number of iterations maxIterations have been reached.

Algorithm 3
Initializing Neural SDF Representation

| | |
|---|---|
| Input: | Neural SDF g(x, t; w), |
| | SDF images \hat{f}(x, t, k) |
| Output: | Initialized neural SDF ĝₒ(x, t; w)∀t ∈ T |

| | |
|---|---|
| 1 | ĝ(x, t; w) = g (x, t; rand(w)) |
| 2 | for iteration < maxIterations do |
| 3 | \| Sample t ~ T |
| 4 | $\vert {L}_{SDF} ← \frac{1}{\vert Ω \vert} {\sum }_{x ∈ Ω} \vert \hat{g} ( x , t ; w ) - \hat{f} ( x , t ) \vert$ |
| 5 | $\vert {L}_{Eikonal} ← \frac{1}{\vert Ω \vert} {\sum }_{x ∈ Ω} \vert {‖ {∇}_{x} \hat{g} ( x , t ; w ) ‖}_{2} - 1 \vert$ |
| 6 | \| L ← L_{SDF} + λL_{Eikonal} |
| 7 | \| w ← w − α∇L |
| 8 | ĝₒ(x, t; w) ← ĝ(x, t; w) |
| 9 | return ĝₒ(x, t; w); |

### IV.B. Training

At this point, the SDF representation ĝₒ(x,t;w) includes motion artifacts present in I_{FBP}. Algorithm 4 optimizes the neural network SDF representation ĝ(x,t;w) to best explain the acquired sinogram r(l,θ) with the minimum total variation in space ∥∇ₓĝ(x,t;w)∥₁ and time |∇₁ĝ(x,t;w)∥₁.

The current neural implicit SDF prediction ĝ(x,t;w) can be converted to a spatiotemporal intensity map Î(x,t) and projected to the sinogram domain via previously described renderer (Section III.) R(Î(x,t),θ). This results in a sinogram estimate \hat{r}(l,θ) used to calculate the sinogram loss L_{Sinogram}—the difference between the current estimate and the acquired sinogram r(l,θ). This loss is combined with the Eikonal constraint L_{Eikonal} and spatial L_{TVS} and temporal TV L_{TVT} loss terms to improve optimization. The optimization is performed until the projection loss L_{Sinogram} decreased below a certain threshold minLoss or maximum number of iterations maxIterations is reached.

### IV.C. Refinement/Exporting

To generate spatiotemporal intensity images from the neural SDF representation, we first convert the neural SDF into a set of explicit SDF images \hat{ƒ}_{□}(x,t,k). This is achieved by sampling the neural SDF ĝ(x,t;w) over a n-dimensional grid at a desired spatial resolution (Algorithm 5). The resulting SDF image is then binarized \hat{B}(x,t,k)=ζ(\hat{ƒ}_{□}(x,t,k)) (Algorithm 6). Binarized images \hat{B}(x,t,k;w) are then used for Refinement, a second pass through the algorithm (starting with Algorithm 2). After Refinement, images are exported and analyzed.

Algorithm 4
Optimizing Neural SDF Representation

| | |
|---|---|
| Input: | Initialized SDF ĝₒ(x, t; w)∀t ∈ T, acquired |
| | sinogram r(l, θ) |
| Output: | Optimized SDF ĝ(x, t; w)∀t ∈ T |

| | |
|---|---|
| 1 | for iteration < maxIterations do |
| 2 | \| Sample t ~ T; |
| 3 | \| Compute projection \hat{r}(l, θ) ← R(Î(x, t), θ(t)); |
| 4 | $\vert {L}_{Sinogram} ← \frac{1}{\vert Ω \vert} {\sum }_{x ∈ Ω} \vert r ( l , θ ) - \hat{r} ( l , θ ) \vert$ |
| 5 | $\vert {L}_{Eikonal} ← \frac{1}{\vert Ω \vert} {\sum }_{x ∈ Ω} \vert {‖ {∇}_{x} {\hat{g}}_{o} ( x , t ; w ) ‖}_{2} - 1 \vert$ |
| 6 | $\vert {L}_{TVS} ← \frac{1}{\vert Ω \vert} {\sum }_{x ∈ Ω} {‖ {∇}_{x} {\hat{g}}_{o} ( x , t ; w ) ‖}_{1}$ |
| 7 | $\vert {L}_{TVT} ← \frac{1}{\vert Ω \vert} {\sum }_{x ∈ Ω} {‖ {∇}_{t} {\hat{g}}_{o} ( x , t ; w ) ‖}_{1}$ |
| 8 | \| L ← L_{Sinogram} + λ₁L_{Eikonal} + λ₂L_{TVS} + λ₃L_{TVT} |
| 9 | \| w ← w − α∇L |
| 10 | \| if L_{Sinogram} < minLoss then |
| 11 | \| \| break |
| 12 | ĝ(x, t; w) ← ĝₒ(x, t; w); |
| 13 | return ĝ(x, t; w); |

### V. Experiments

### V.A. Implementation Details

The image resolution used during training is set to n=128 with upsampling factor kₛₐₘₚ=2. For optimal performance, we used Fₘₐₓ=3.0 and μ=50. In Algorithm 1, we sampled τ such that |τ|=0.02*|T| and set κ=3. For Algorithms 3 & 4 the learning rate is α=1×10⁻⁵ and decayed by a factor of 0.95 every 200 steps. The optimization procedure is run with maxIterations=5000, minLoss=0.08, and λ=0.1 and performed 5 times, each with a different random seed for the neural network. For best results, we trained with minibatch 1=20. As described below, we observed optimal results with λ₁=0.1, λ₂=0.5, and λ₃=0.5. All experiments are performed using a 2D parallel beam CT geometry of a single foreground class (i.e., K=1) except for the multi-object study. Optimizing DiFiR-CT takes ˜30 mins on a single NVIDIA 1080Ti GPU.

### V.B. Evaluation of DiFiR-CT Parameters

First, we simulated a simple cause of motion corruption: translation of a circle duringimaging. This is intended to mimic motion of a coronary artery. A circle with a diameter 25% of the size of the image is placed that same distance (25% of the image) from the center and angularly translated during the acquisition. We used this toy problem to explore the impact of λ₁, λ₂, and Fₘₐₓ on the ability of DiFiR-CT to faithfully reconstruct a moving object. λ₁ and λ₂ are the weight of spatial and temporal total variation in the cost function used to supervise learning and Fₘₐₓ is the maximum Fourier coefficient available for representation of the temporal changes.

The impact of these parameters on DiFiR-CT is evaluated via parameter sweeps. First, λ₁ and λ₂ are varied (0, 0.05, 0.1, 0.15, 0.3, 0.5, 0.9, 1.5, 3.0, 5.0, 10.0) with Fₘₐₓ set to 3.0. Then, Fₘₐₓ is varied from 0 to 10.0 with λ₁=0.5 and λ₂=0.5.

We evaluated a range of angular displacements per gantry rotation (0, 1, 5, 10, 20, 40, 70, 100, 120, 150, and 200 degrees per gantry rotation). DiFiR-CT results are compared to both the ground-truth vessel image as well as the FBP result using the metrics described below.

### V.C. Evaluation of Noise on DiFiR-CT

The impact of noise in the acquired sinogram on the quality of DiFiR-CT reconstruction is evaluated using imaging data obtained of a circle undergoing an angular displacement of 100 degrees. Poisson-distributed quanta-counting noise is added to the acquired sinogram and the incident x-ray beam intensity is modulated to create images with a range of contrast-to-noise ratios (from 3 to 40). Five different noise realizations are created and DiFiR-CT and FBP results are compared to the ground-truth using the metrics described below.

### V.D. Multi-Object DiFiR-CT and Impact of Initial Segmentation

As outlined above, a key step in the DiFiR-CT framework is the initialization of the SDF map of scene. Above, we described a Gaussian Mixture Model (GMM) approach that is solely based on the intensity histogram in I_{FBP}(x,t). However, when applied to a scene with multiple moving objects, each with different attenuations, the GMM may fail to differentiate objects based solely on the intensity distribution. FIG. 10 shows a failure of the GMM approach when analyzing the FBP reconstruction of two moving dots with two different attenuations (top=0.7, bottom=0.2). GMM identifies two intensity values of interest by evaluating the distribution of pixel intensities. However, the second object identified is the motion artifact of the brighter dot. Therefore, we refined the segmentation by providing some spatial information. A second approach, SegSI, uses a Region-Of-Interest (ROI) to guide thresholding-based segmentation. A bounding box is defined to only contain one moving dot such that we assigned one individual class to each box. In the box, we defined an intensity threshold=γ×Iₘₐₓ where Iₘₐₓ is the maximum intensity in the scene in each box to capture the real object. γ=0.7 is set empirically. In all other respects, DiFiR-CT remained the same such that we could illustrate the improvement associated with an improved initialization.

### V.E. Imaging of Nonrigid, Heart-Like Motion

To evaluate the utility of DiFiR-CT during non-rigid motion, we modeled heart-like motion using an ellipse with time-varying axes. In addition to static imaging, cine CT imaging can be used to evaluate heart size and dynamics. Therefore, we evaluated two imaging approaches: 1) Single-gantry rotation imaging where a sinogram spanning 360 degrees is acquired during which the ellipse diameter changes. 2) Full heart cycle imaging where multiple (typically 4-5) gantry rotations are obtained spanning the entire period of the heart-like motion. Of note, DiFiR-CT readily incorporate multiple rotation data into the reconstruction framework without modification. In this scenario, DiFiR-CT results are compared to FBP reconstructions centered at the same temporal position.

### V.F. Imaging of Complex Deformations

Lastly, we demonstrate the ability of DiFiR-CT to reconstruct scenes with complex topological change. To do so, we created a complex scene where the letter “A” transforms into “B”, then into “C”, then back to “B” and “A”. Without changes to any DiFiR-CT parameters (spatial/temporal weighting and Fourier coefficients), DiFiR-CT results are compared to FBP imaging.

### V.G. Metrics to Evaluate Image Quality

DiFiR-CT is compared to conventional FBP images using mean-square error (MSE) and the foreground's Dice coefficient where FBP images are thresholded using half of the foreground intensity. We did not compare DiFiR-CT to motion correction methods. This is motivated by the fact that the improvement obtained via correction depends on the suitability of the motion model in the correction scheme to the motion present in the acquired data. Given that current correction methods have been designed for clinical scenarios, our motion scenarios are not expected to represent realistic use cases. For DiFiR-CT, we report the median accuracy of 5 independent optimizations, where each optimization is initialized with a different random seed.

### VI. Results

### VI.A. Moving Coronary Vessel Imaging

As shown by the images and metrics in FIG. 11, DiFiR-CT accurately reconstructed images of a small circle while FBP images showed significant artifacts (λ₁=0.5, λ₂=0.5, and Fₐₓ=3.0). Low MSE and high (>0.9) Dice is maintained for angular displacements of up to 150° during data acquisition. FIG. 11 also illustrates how the DiFiR-CT reconstruction is impacted by displacements >150°. While the shape of the circle is preserved, increased MSE and decreased Dice occur primarily due to reconstruction of the circle at the incorrect temporal position.

### VI.B. Evaluation of DiFiR-CT Parameters

DiFiR-CT reconstruction of a circle with translation=100° per gantry rotation is used to evaluate the robustness of DiFiR-CT to changes in λ₁, λ₂, and Fₘₐₓ. FIG. 12A illustrates the minor impact of changing the strength of spatial (λ₁) and temporal (λ₂) regularization while maintaining Fₘₐₓ=3.0. Accurate reconstruction is achieved over a wide range of regularization strength. FIG. 12B illustrates the performance of DiFiR-CT with λ₁=0.5 and λ₂=0.5 when varying Fₘₐₓ. Decreasing Fₘₐₓ limits the temporal evolution that can be represented by the neural implicit approach. However, we observed robust DiFiR-CT reconstruction performance even at low Fₘₐₓ values. Reconstruction accuracy decreased at high Fₘₐₓ. This suggests that introduction of high frequency coefficients can result in overfitting.

### VI.C. DiFiR-CT Reconstruction of Motion and Noise

Without modification of the parameters identified above (λ₁=0.5, λ₂=0.5, and Fₘₐₓ=3.0), DiFiR-CT successfully reconstructed images across a range of contrast-to-noise values in the acquired sinogram. FIG. 13 illustrates the high image quality of DiFiR-CT as well as the loss of image quality using the FBP approach. Despite significant object motion and low (≤5) CNR, DiFiR-CT maintained MSE≤0.0031 and Dice≥0.91 while FBP results are significantly worse in this scenario (MSE≥0.02, Dice≤0.05).

### VI.D. Multi-Object DiFiR-CT and Impact of Initial Segmentation

Reconstruction with DiFiR-CT-SegGMM is limited when Δθ>60 degrees (FIG. 14). In contrast, DiFiR-CT-SegSI maintained high-quality motion-corrected reconstructions for all Δθ and achieved low RSME (<0.028) and high (>0.89) DICE for Δθ up to 160 degrees. This illustrates several key features of the DiFiR-CT framework. First, solving for two intensities illustrates the ability of DiFiR-CT to be extended to multi-intensity scenes. Second, a spatially-naive segmentation can lead to limited performance. However, simple modifications such as the use of a ROI can significantly improve the result. For example, one potential use of DiFiR-CT may correct motion artifacts of a particular object that the user selects via an ROI.

### VI.E. Imaging Nonrigid, Heart-Like Motion with DiFiR-CT

Without modification of the parameters identified above (λ₁=0.5, λ₂=0.5, and Fₘₐₓ=3.0), DiFiR-CT successfully reconstructed images of the heart-like motion for both single gantry and full heart cycle imaging. Relative to FBP, DiFiR-CT improved imaging metrics when imaging an ellipse with changing dimensions. FIG. 15 illustrates these differences. The change in axes dimension is shown by the left two columns (temporal reformats along the x- and y-direction). Metrics of image quality (MSE and Dice) are shown on the right. For full heart cycle imaging with beating velocity=3.0, DiFiR-CT decreased MSE (FBP: median 0.005, IQR 0.003-0.009, DiFiR-CT: median 0.001, IQR 0.001-0.002). Further, DiFiR-CT increased the percentage of frames with MSE<0.005 from 46% (FBP) to 100%. DiFiR-CT also improved Dice (FBP: median 0.84, IQR 0.73-0.88 to DiFiR-CT: median 0.96, IQR 0.95-0.97).

### VI.F. Imaging Complex Topology Changes with DiFiR-CT

Without modification of the DiFiR-CT framework or parameters (λ₁=0.5, λ₂=0.5, and Fₘₐₓ=3.0), DiFiR-CT successfully reconstructed data acquired during a complex letter warping scene. FIG. 16 shows five frames of the deformation with the groundtruth (top) as well as FBP (middle) and DiFiR-CT (bottom) reconstructions. DiFiR-CT significantly reduced the severity of artifacts created when using FBP. The plot of MSE and Dice scores as a function of time further illustrate the improvement. DiFiR-CT decreased error as measured via MSE (FBP: median 0.011, IR 0.008-0.019 to DiFiR-CT: median 0.007, IQR 0.004-0.013). Further, DiFiR-CT increased the percentage of the frames with MSE<0.005 from 15.2% to 34%. DICE scores also improved with DiFiR-CT (FBP: median 0.78, IQR 0.66-0.85, DiFiR-CT: median 0.86, IQR 0.80-0.91). The percentage of frames with Dice >0.85 increased from 25.8% for FBP to 58.8% with DiFiR-CT.

DiFiR-CT combines implicit representation of an object by a signed distance function (SDF) with differentiable rendering to successfully enable time-resolved imaging free from motion artifacts despite data acquisition occurring during motion. DiFiR-CT takes advantage of several important features of SDFs—namely, that they represent movement of a boundary as a spatially and temporally smooth evolution. DiFiR-CT represents the scene as SDFs which evolve over time using an implicit representation; in this case, a neural network. Differentiable rendering is used to improve the estimate of the scene by comparing the observed CT data with the SDF-based representation in the sinogram domain. The framework also enables additional regularization such as penalizing deviations from the Eikonal constraint and minimizing spatial and temporal variations. We demonstrated the utility of DiFiR-CT in three different imaging scenarios without changes to the architecture. Specifically, DiFiR-CT is readily applied to objects with different motions as well as data spanning one or more than one gantry rotation. These cases highlight how DiFiR-CT can be used to accurately reconstruct objects undergoing 1) translation, 2) heartbeat-like affine changes in diameter, and 3) complex topological changes (warping of letters). This flexibility is facilitated by the fact that DiFiR-CT does not utilize an explicit motion model.

In some embodiments of the disclosed technology, DiFiR-CT may perform accurate reconstructions even with a fairly limited set of Fourier coefficients. For example, this is due to the beneficial fact that SDFs used to parameterize the reconstruction problem evolve smoothly over space and time. In some embodiments of the disclosed technology, reconstruction performance may decrease if Fₘₐₓ increased. This highlights the benefit of limiting the complexity of the solution via Fₘₐₓ. An added advantage is that this hyperparameter has a simple physical interpretation, the bandwidth of the motion, which may facilitate its selection when applying DiFiR-CT to novel applications. In some embodiments of the disclosed technology, additional constraints such as preservation of mass may improve imaging in certain scenarios.

In one implementation, DiFiR-CT may be used to image one or two foreground objects with an empty background. In another implementation, the background class may be used to represent the position and intensity of static objects. In some implementations, DiFiR-CT may resolve scenes with more complex background intensity.

Some embodiments illustrate DiFiR-CT results without modification of parameters λ₁, λ₂, and Fₘₐₓ. However, the optimal choice of these parameters is expected to vary depending on the specific imaging scenarios. Further, the rotation speed of CT systems can depend on both the design of the system and the clinical protocol. Evaluating the optimal combination of parameters is left for future work where specific scenarios are evaluated.

Some embodiments illustrate how DiFiR-CT can seamlessly reconstruct data acquired using two common CT acquisition approaches (single and multiple gantry rotation imaging). Specifically, DiFiR-CT reconstructed imaging data spanning multiple gantry rotations simultaneously without explicit labeling of timepoints or the need for specific, complementary images. This is significantly different than approaches such as FBP which reconstruct each frame independently. As a result, in addition to improving single gantry reconstruction, DiFiR-CT also has the potential to improve reconstruction quality of each frame acquired during a multiple gantry acquisition by leveraging additional temporal information. While acquiring additional temporal information (beyond the typical half- or single gantry rotation) increases the dose delivered to the patient, it may enable DiFiR-CT to resolve the dynamics of objects that have been significantly hampered by imaging artifacts.

Some embodiments quantify DiFiR-CT image quality relative to the groundtruth and conventional FBP reconstruction instead of direct comparison to previously-reported motion-correction approaches. Some embodiments do not to compare DiFiR-CT to techniques specially crafted for certain imaging scenarios such as translation of a coronary artery because the performance of motion correction approaches to depend significantly on the specifics of the application. For example, motion-correction approaches to accurately reconstruct scenes when the object motion agrees with the method's motion model. However, limited performance may occur if the object motion is different from the motion model.

In some embodiments, DiFiR-CT uses a single-source 2D parallel beam geometry to simplify interpretability of our findings. However, the 4D problem is a major motivation behind our use of a neural representation for the distance field. By storing the representation as a neural network, DiFiR-CT implemented based on some embodiments does not need to represent the number of pixels/voxels in the final reconstructed image. For example, in 2D examples, DiFiR-CT implemented based on some embodiments reconstructs 720 128×128 images (11.8 million pixels) using neural representations with ˜200 k weights. Practically speaking, this approach has the advantage that encoding a 3D field simply involves adding one more channel/input dimension to our neural representation. DiFiR-CT implemented based on some embodiments may determine the exact architecture needed to accurately represent a 3D distance field without suffering from the same scalability challenges faced by voxel-based representations.

Some embodiments may solve for multiple foreground classes when an object has a uniformity intensity which does not change over time.

The disclosed technology can be implemented in some embodiments to complement previously described motion correction methods that operate on intensity values by providing accurate boundary position information. In some embodiments, when solving for multiple moving objects, accurate optimization begins to depend, to a greater extent, on the initial segmentation of the filtered backprojection image and initialization of the algorithm. The use of a bounding box may be limited in certain clinical applications and is selected as a simple refinement to our process. In some embodiments, more advanced initializations, such as semantic segmentations provided by experts or automated methods, may serve as robust initializations. However, the availability of such segmentations may depend on the clinical application.

Some embodiments utilize SIRENs as the neural network representation for the spatiotemporal SDF.

In some embodiments, dual-source systems and fan- or cone-beam geometries may change the relationship between object motion and acquired CT data. DiFiR-CT may also improve image quality in these scenarios.

In some embodiments, a novel reconstruction framework, DiFiR-CT, can be used to reconstruct CT imaging data acquired during object motion in a time-resolved manner free from motion artifacts. The disclosed technology can be implemented in some embodiments to leverage a neural implicit scheme without requiring a prior motion models or explicit motion estimation. Representing moving boundaries using a signed distance metric and neural implicit framework enables ‘analysis-by-synthesis’ to identify a solution consistent with the observed sinogram as well as spatial and temporal consistency constraints.

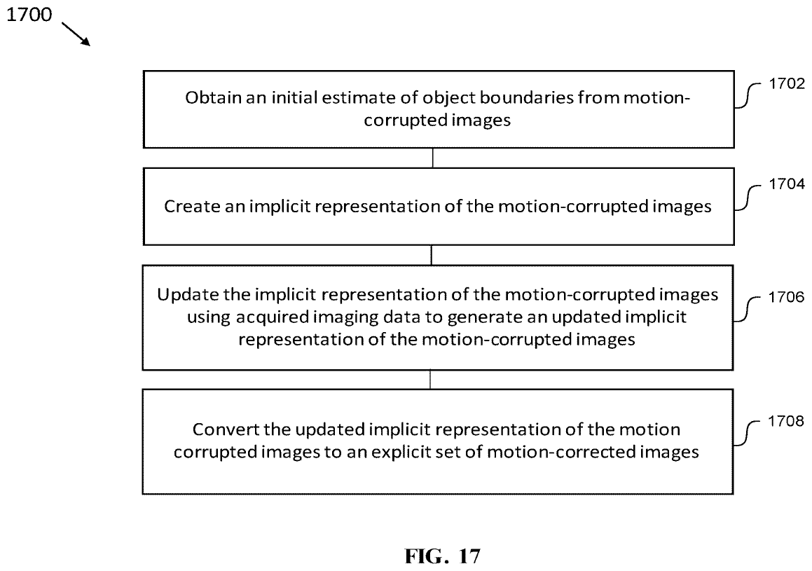

FIG. 17 shows an example image reconstruction method 1700 based on some embodiments of the disclosed technology.

In some implementations, the method 1700 includes, at 1702, obtaining an initial estimate of object boundaries from motion-corrupted images, at 1704, creating an implicit representation of the motion-corrupted images, at 1706, updating the implicit representation of the motion-corrupted images using acquired imaging data to generate an updated implicit representation of the motion-corrupted images, and at 1708, converting the updated implicit representation of the motion corrupted images to an explicit set of motion-corrected images.

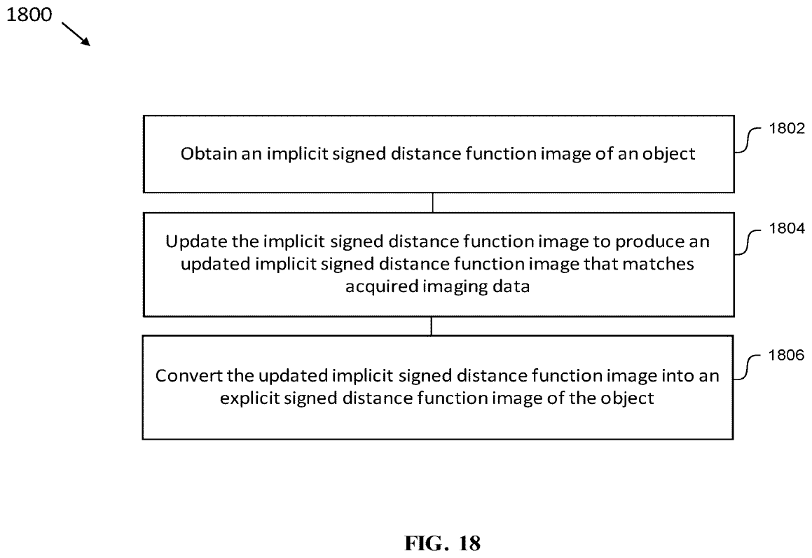

FIG. 18 shows an example image reconstruction method 1800 based on some embodiments of the disclosed technology.

In some implementations, the method 1800 includes, at 1802, obtaining an implicit signed distance function image of an object, at 1804, updating the implicit signed distance function image to produce an updated implicit signed distance function image that matches acquired imaging data, and at 1806, converting the updated implicit signed distance function image into an explicit signed distance function image of the object.

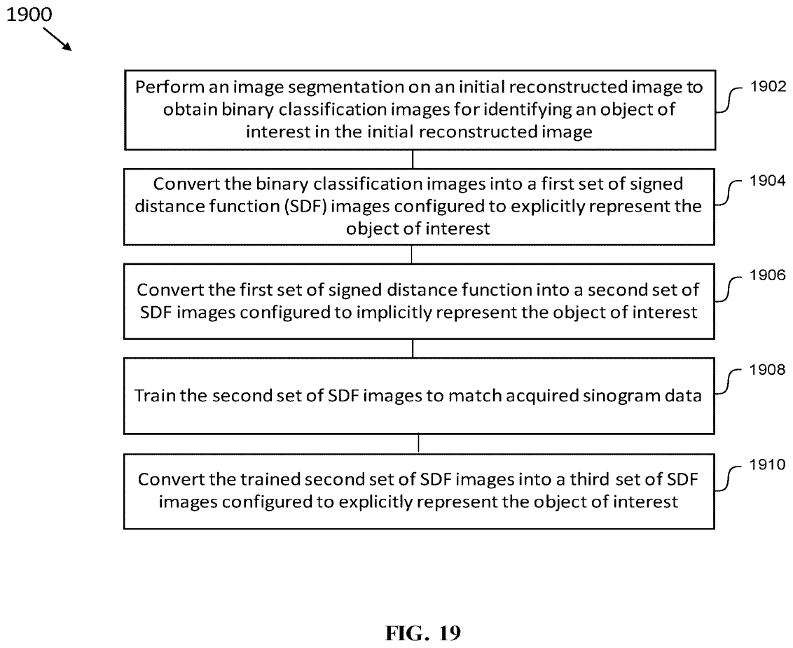

FIG. 19 shows an example image reconstruction method 1900 based on some embodiments of the disclosed technology.

In some implementations, the method 1900 includes, at 1902, performing an image segmentation on an initial reconstructed image to obtain binary classification images for identifying an object of interest in the initial reconstructed image, at 1904, converting the binary classification images into a first set of signed distance function (SDF) images configured to explicitly represent the object of interest, at 1906, converting the first set of signed distance function into a second set of SDF images configured to implicitly represent the object of interest, at 198, training the second set of SDF images to match acquired sinogram data, and at 1910, converting the trained second set of SDF images into a third set of SDF images configured to explicitly represent the object of interest. In one example, the image segmentation includes an intensity-based segmentation.

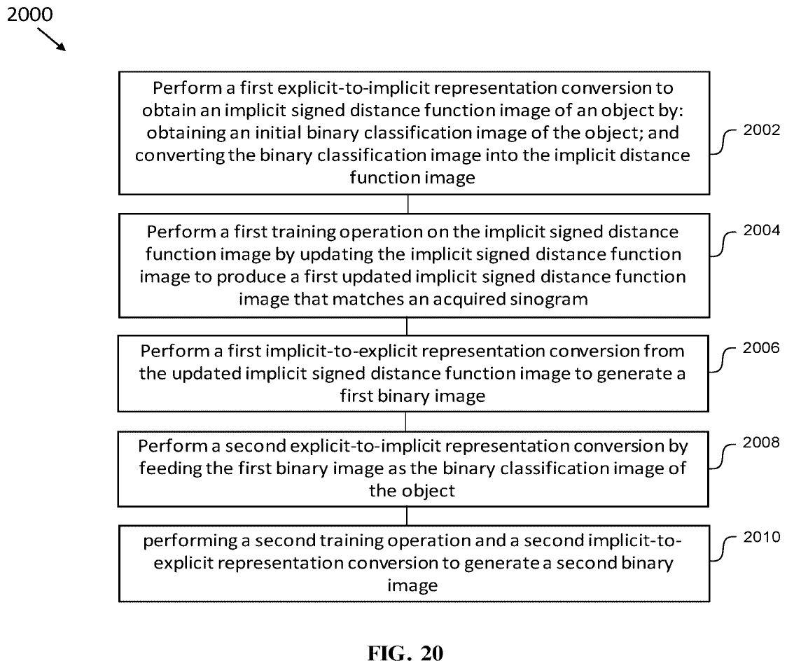

FIG. 20 shows an example image reconstruction method 2000 based on some embodiments of the disclosed technology.

In some implementations, the method 2000 includes, at 2002, performing a first explicit-to-implicit representation conversion to obtain an implicit signed distance function image of an object by: obtaining an initial binary classification image of the object; and converting the binary classification image into the implicit distance function image, at 2004, performing a first training operation on the implicit signed distance function image by updating the implicit signed distance function image to produce a first updated implicit signed distance function image that matches an acquired sinogram, at 2006, performing a first implicit-to-explicit representation conversion from the updated implicit signed distance function image to generate a first binary image, at 2008, performing a second explicit-to-implicit representation conversion by feeding the first binary image as the binary classification image of the object, and at 2010, performing a second training operation and a second implicit-to-explicit representation conversion to generate a second binary image.

Therefore, various implementations of features of the disclosed technology can be made based on the above disclosure, including the examples listed below.

Example 1. An image reconstruction method, comprising: obtaining an initial estimate of object boundaries from motion-corrupted images; creating an implicit representation of the motion-corrupted images; updating the implicit representation of the motion-corrupted images using acquired imaging data to generate an updated implicit representation of the motion-corrupted images; and converting the updated implicit representation of the motion corrupted images to an explicit set of motion-corrected images.

Example 2. The method of example 1, wherein obtaining the initial estimate of object boundaries from motion-corrupted images includes performing an image segmentation to obtain binary classification images. In one example, the image segmentation includes an intensity-based segmentation.

Example 3. The method of example 1, wherein the implicit representation of the motion includes a distance representation. In one example, the distance representation includes a signed distance function image. In another example, the distance representation includes an unsigned distance function image.

Example 4. The method of example 3, wherein the acquired imaging data includes acquired sinogram data associated with the motion corrupted images.

Example 5. The method of example 4, wherein updating the implicit representation of the motion corrupted images using acquired imaging data includes updating the SDF images to produce updated SDF images that match the acquired sinogram data.

Example 6. The method of example 5, wherein converting the updated implicit representation of the motion corrupted images to the explicit set of motion-corrected images includes sampling the updated implicit representation over a grid at a predetermined spatial resolution to generate spatiotemporal intensity images.

Example 7. An image reconstruction method, comprising: obtaining an implicit signed distance function image of an object; updating the implicit signed distance function image to produce an updated implicit signed distance function image that matches acquired imaging data; and converting the updated implicit signed distance function image into an explicit signed distance function image of the object.

Example 8. The method of example 7, wherein obtaining the implicit signed distance function image of the object includes: generating, from a filtered back projection image of the object, a binary classification image of the object; and converting the binary classification image into the implicit distance function image.

Example 9. The method of example 8, wherein the binary classification image is generated by: identifying the object in an image reconstructed through a filtered back projection by performing an image segmentation; and encoding, using a signed distance function, a segmentation image of the object that is obtained by performing the image segmentation. In one example, the image segmentation includes an intensity-based segmentation.

Example 10. The method of example 7, wherein the implicit signed distance function image is generated using a neural network.

Example 11. The method of example 7, wherein the acquired imaging data includes acquired sinogram data.

Example 12. The method of example 11, wherein the acquired sinogram data includes data that is acquired by a computed tomography (CT) scanner.

Example 13. The method of example 7, wherein converting the updated implicit signed distance function image into the explicit signed distance function image of the object includes sampling the updated implicit signed distance function image over a grid at a predetermined spatial resolution to generate a spatiotemporal intensity image of the object.

Example 14. An image reconstruction method, comprising: performing an image segmentation on an initial reconstructed image to obtain binary classification images for identifying an object of interest in the initial reconstructed image; converting the binary classification images into a first set of signed distance function (SDF) images configured to explicitly represent the object of interest; converting the first set of signed distance function into a second set of SDF images configured to implicitly represent the object of interest; training the second set of SDF images to match acquired sinogram data; and converting the trained second set of SDF images into a third set of SDF images configured to explicitly represent the object of interest. In one example, the image segmentation includes an intensity-based segmentation.

Example 15. The method of example 14, further comprising performing a filtered back projection (FBP) on the object of interest to obtain the initial reconstructed image of the object

Example 16. The method of example 14, wherein the second set of SDF are represented by a neural network.

Example 17. The method of example 16, wherein training the second set of SDF images comprises: converting the second set of SDF images to a spatiotemporal intensity map and projecting to a sinogram domain to determine a sinogram estimate; determining a difference between the sinogram estimate and the acquired sinogram data; and updating the second set of SDF images based on the difference by backpropagating the difference through neural representations of the SDF images.

Example 18. The method of example 14, wherein the binary classification images represent pixels of each label across time.

Example 19. The method of example 14, wherein the acquired sinogram data includes data that is acquired by a computed tomography (CT) scanner.

Example 20. The method of example 14, wherein the acquired sinogram data includes an attenuation accumulated by x-rays traversing from a light source to a specific detector position associated with the object of interest.

Example 21. The method of example 14, wherein converting the trained second set of SDF images into the third set of SDF images includes sampling the trained second set of SDF images over a grid at a predetermined spatial resolution.

Example 22. The method of example 14, further comprising creating occupancy images by binarizing the third set of SDF images.

Example 23. An image reconstruction method, comprising: performing a first explicit-to-implicit representation conversion to obtain an implicit signed distance function image of an object by: obtaining an initial binary classification image of the object; and converting the binary classification image into the implicit distance function image; performing a first training operation on the implicit signed distance function image by updating the implicit signed distance function image to produce a first updated implicit signed distance function image that matches an acquired sinogram; performing a first implicit-to-explicit representation conversion from the updated implicit signed distance function image to generate a first binary image; performing a second explicit-to-implicit representation conversion by feeding the first binary image as the binary classification image of the object; and performing a second training operation and a second implicit-to-explicit representation conversion to generate a second binary image.

Example 24. The method of example 23, wherein the initial binary classification image of the object is obtained from a filtered back projection image of the object.

Example 25. The method of example 23, wherein the second training operation includes updating an implicit signed distance function image obtained by performing the second explicit-to-implicit representation conversion to produce a second updated implicit signed distance function image that matches an acquired sinogram.

Example 26. The method of example 23, wherein the first implicit-to-explicit representation conversion includes sampling the updated implicit signed distance function image over a grid at a predetermined spatial resolution.

Example 27. The method of example 26, wherein the first binary image is generated by binarizing the sampled updated implicit signed distance function image.

Example 28. The method of example 23, wherein the initial binary classification image is generated by: identifying the object in an image reconstructed through a filtered back projection by performing an image segmentation; and encoding, using a signed distance function, a segmentation image of the object that is obtained by performing the image segmentation.

Example 29. A system for image reconstruction, comprising a memory and a processor, wherein the processor reads code from the memory and implements a method recited in any of examples 1 to 28.

Performing the operations that are disclosed in this patent document, including those in Examples 1-28, can include operating an imaging device, such as but not limited to a CT or an MRI imaging device, to obtain imaging data of a patient. The imaging data can be received and processed by a processor that can be at a remote location, or can be close to or even part of the imaging device. The processor can execute program code that it retrieves from a tangle storage medium to perform the operations disclosed herein. The processor can further produce improved images that can be presented on a display.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1-6. (canceled)

7. An image reconstruction method, comprising:
obtaining an implicit signed distance function image of an object;
updating the implicit signed distance function image to produce an updated implicit signed distance function image that matches acquired imaging data; and
converting the updated implicit signed distance function image into an explicit signed distance function image of the object.

8. The method of claim 7, wherein obtaining the implicit signed distance function image of the object includes: generating, from a filtered back projection image of the object, a binary classification image of the object; and converting the binary classification image into the implicit distance function image.

9. The method of claim 8, wherein the binary classification image is generated by: identifying the object in an image reconstructed through a filtered back projection by performing an image segmentation; and
encoding, using a signed distance function, a segmentation image of the object that is obtained by performing the image segmentation.

10. The method of claim 7, wherein the implicit signed distance function image is represented by a neural network.

11. The method of claim 7, wherein the acquired imaging data includes acquired sinogram data.

12. The method of claim 11, wherein the acquired sinogram data includes data that is acquired by a computed tomography (CT) scanner.

13. The method of claim 7, wherein converting the updated implicit signed distance function image into the explicit signed distance function image of the object includes sampling the updated implicit signed distance function image over a grid at a predetermined spatial resolution to generate a spatiotemporal intensity image of the object.

14. An image reconstruction method, comprising:
performing an image segmentation on an initial reconstructed image to obtain binary classification images for identifying an object of interest in the initial reconstructed image;
converting the binary classification images into a first set of signed distance function (SDF) images configured to explicitly represent the object of interest;
converting the first set of signed distance function into a second set of SDF images configured to implicitly represent the object of interest;
training the second set of SDF images to match acquired sinogram data; and
converting the trained second set of SDF images into a third set of SDF images configured to explicitly represent the object of interest.

15. The method of claim 14, further comprising performing a filtered back projection (FBP) on the object of interest to obtain the initial reconstructed image of the object.

16. The method of claim 14, wherein the second set of SDF images are represented by a neural network.

17. The method of claim 16, wherein training the second set of SDF images comprises: converting the second set of SDF images to a spatiotemporal intensity map and projecting to a sinogram domain to determine a sinogram estimate;
determining a difference between the sinogram estimate and the acquired sinogram data; and
updating the second set of SDF images based on the difference by backpropagating the difference through neural representations of the SDF images.

18. The method of claim 14, wherein the binary classification images represent pixels of each label across time.

19. The method of claim 14, wherein the acquired sinogram data includes data that is acquired by a computed tomography (CT) scanner.

20. The method of claim 14, wherein the acquired sinogram data includes an attenuation accumulated by x-rays traversing from a light source to a specific detector position associated with the object of interest.

21. The method of claim 14, wherein converting the trained second set of SDF images into the third set of SDF images includes sampling the trained second set of SDF images over a grid at a predetermined spatial resolution.

22. The method of claim 14, further comprising creating occupancy images by binarizing the third set of SDF images.

23. An image reconstruction method, comprising:
performing a first explicit-to-implicit representation conversion to obtain an implicit signed distance function image of an object by: obtaining an initial binary classification image of the object; and converting the binary classification image into the implicit distance function image;
performing a first training operation on the implicit signed distance function image by updating the implicit signed distance function image to produce a first updated implicit signed distance function image that matches an acquired sinogram;
performing a first implicit-to-explicit representation conversion from the updated implicit signed distance function image to generate a first binary image;
performing a second explicit-to-implicit representation conversion by feeding the first binary image as the binary classification image of the object; and
performing a second training operation and a second implicit-to-explicit representation conversion to generate a second binary image.

24. The method of claim 23, wherein the initial binary classification image of the object is obtained from a filtered back projection image of the object.

25. The method of claim 23, wherein the second training operation includes updating an implicit signed distance function image obtained by performing the second explicit-to-implicit representation conversion to produce a second updated implicit signed distance function image that matches an acquired sinogram.

26. The method of claim 23, wherein the first implicit-to-explicit representation conversion includes sampling the updated implicit signed distance function image over a grid at a predetermined spatial resolution.

27. The method of claim 26, wherein the first binary image is generated by binarizing the sampled updated implicit signed distance function image.

28. The method of claim 23, wherein the initial binary classification image is generated by:
identifying the object in an image reconstructed through a filtered back projection by performing an image segmentation; and
encoding, using a signed distance function, a segmentation image of the object that is obtained by performing the image segmentation.

29. (canceled)
